(19) United States
(12) Patent Application Publication
(10) Pub. No.: US 2011/0009863 A1
(43) Pub. Date: Jan. 13, 2011

(54) Shaft Constructions for Medical Devices with an Articulating Tip

(75) Inventors: Stanislaw Marczyk, Stratford, CT (US); Russell Pribanic, New Milford, CT (US)

Correspondence Address: Tyco Healthcare Group LP;d/b/a Covidien, 555 Long Wharf Drive, Mail Stop 8-N1, Legal Department, New Haven, CT, 06511, US

(73) Assignee: Tyco Healthcare Group LP

(21) Appl. No.: 12/718,131

(22) Filed: Mar. 5, 2010

Related U.S. Application Data
(60) Provisional application No. 61/224,485, filed on Jul. 10, 2009.
(60) Provisional application No. 61/224,486, filed on Jul. 10, 2009.
(60) Provisional application No. 61/224,484, filed on Jul. 10, 2009.
(60) Provisional application No. 61/249,048, filed on Oct. 6, 2009.

(51) Int. Cl. A61B 18/04

(52) U.S. Cl. 606/51

(57) ABSTRACT

An endoscopic surgical instrument for sealing tissue includes an end effector having a pair of jaw members adapted to connect to a source of electrosurgical energy. At least one jaw member is movable relative to the other between an open configuration and a closed configuration for grasping tissue. A handle is movable to induce motion in the end effector between the open and closed configurations. An elongated shaft defines a longitudinal axis and is coupled between the end effector and the handle. The shaft includes a plurality of links arranged such that neighboring links engage one another across a pair of edges to maintain the end effector in an aligned configuration with respect to the longitudinal axis. Each of the edges is spaced laterally from the longitudinal axis. The neighboring links may pivot about the rotational edges to move the end effector to an articulated configuration.

### CROSS REFERENCE TO RELATED APPLICATIONS

The present application claims the benefit of and priority to U.S. Provisional Application Ser. No. 61/224,485 filed on Jul. 10, 2009, U.S. Provisional Application Ser. No. 61/224,486 filed on Jul. 10, 2009, U.S. Provisional Application Ser. No. 61/224,484 filed on Jul. 10, 2009, and U.S. Provisional Application Ser. No. 61/249,048 filed on Oct. 6, 2009. The entire content of each of these Applications is incorporated herein by reference.

### BACKGROUND

The present disclosure relates to an electrosurgical forceps. More particularly, the present disclosure relates to an endoscopic electrosurgical forceps for sealing and/or cutting tissue utilizing an elongated, generally flexible and articulating shaft.

### TECHNICAL FIELD

Electrosurgical forceps utilize both mechanical clamping action and electrical energy to effect hemostasis by heating the tissue and blood vessels to coagulate, cauterize and/or seal tissue. As an alternative to open forceps for use with open surgical procedures, many modern surgeons use endoscopes and endoscopic instruments for remotely accessing organs through smaller, puncture-like incisions. As a direct result thereof, patients tend to benefit from less scarring and reduced healing time.

Generally, endoscopic surgery involves incising through body walls for example, viewing and/or operating on the ovaries, uterus, gall bladder, bowels, kidneys, appendix, etc. There are many common endoscopic surgical procedures, including arthroscopy, laparoscopy (pelviscopy), gastroentroscopy and laryngobronchoscopy, just to name a few. Typically, trocars are utilized for creating the incisions through which the endoscopic surgery is performed.

Trocar tubes or cannula devices are extended into and left in place in the abdominal wall to provide access for endoscopic surgical tools. A camera or endoscope is inserted through a relatively large diameter trocar tube which is generally located at the naval incision, and permits the visual inspection and magnification of the body cavity. The surgeon can then perform diagnostic and therapeutic procedures at the surgical site with the aid of specialized instrumentation, such as, forceps, cutters, applicators, and the like which are designed to fit through additional cannulas. Thus, instead of a large incision (typically 12 inches or larger) that cuts through major muscles, patients undergoing endoscopic surgery receive more cosmetically appealing incisions, between 5 and 10 millimeters in size. Recovery is, therefore, much quicker and patients require less anesthesia than traditional surgery. In addition, because the surgical field is greatly magnified, surgeons are better able to dissect blood vessels and control blood loss.

In continuing efforts to reduce the trauma of surgery, interest has recently developed in the possibilities of performing procedures to diagnose and surgically treat a medical condition without any incision in the abdominal wall by using a natural orifice (e.g., the mouth or anus) to access the target tissue. Such procedures are sometimes referred to as endoluminal procedures, transluminal procedures, or natural orifice transluminal endoscopic surgery (“NOTES”). Although many such endoluminal procedures are still being developed, they generally utilize a flexible endoscope instrument or flexible catheter to provide access to the tissue target tissue. Endoluminal procedures have been used to treat conditions within the lumen including for example, treatment of gastroesophageal reflux disease in the esophagus and removal of polyps from the colon.

In some instances, physicians have gone beyond the luminal confines of the gastrointestinal tract to perform intra-abdominal procedures. For example, using flexible endoscopic instrumentation, the wall of the stomach can be punctured and an endoscope advanced into the peritoneal cavity to perform various procedures. Using such endoluminal techniques, diagnostic exploration, liver biopsy, cholecystectomy, splenectomy, and tubal ligation have reportedly been performed in animal models. After the intra-abdominal intervention is completed, the endoscopic instrumentation is retracted into the stomach and the puncture closed. Other natural orifices, such as the anus or vagina, may also allow access to the peritoneal cavity.

As mentioned above, many endoscopic and endoluminal surgical procedures typically require cutting or ligating blood vessels or vascular tissue. However, this ultimately presents a design challenge to instrument manufacturers who must attempt to find ways to make endoscopic instruments that fit through the smaller cannulas. Due to the inherent spatial considerations of the surgical cavity, surgeons often have difficulty suturing vessels or performing other traditional methods of controlling bleeding, e.g., clamping and/or tying-off transected blood vessels. By utilizing an endoscopic electrosurgical forceps, a surgeon can either cauterize, coagulate/desiccate and/or simply reduce or slow bleeding simply by controlling the intensity, frequency and duration of the electrosurgical energy applied through the jaw members to the tissue. Most small blood vessels, i.e., in the range below two millimeters in diameter, can often be closed using standard electrosurgical instruments and techniques. However, if a larger vessel is ligated, it may be necessary for the surgeon to convert the endoscopic procedure into an open-surgical procedure and thereby abandon the benefits of endoscopic surgery. Alternatively, the surgeon can seal the larger vessel or tissue utilizing specialized vessel sealing instruments.

It is thought that the process of coagulating vessels is fundamentally different than electrosurgical vessel sealing. For the purposes herein, “coagulation” is defined as a process of desiccating tissue wherein the tissue cells are ruptured and dried. “Vessel sealing” or “tissue sealing” is defined as the process of liquefying the collagen in the tissue so that it reforms into a fused mass. Coagulation of small vessels is sufficient to permanently close them, while larger vessels need to be sealed to assure permanent closure. Moreover, coagulation of large tissue or vessels results in a notoriously weak proximal thrombus having a low burst strength whereas tissue seals have a relatively high burst strength and may be effectively severed along the tissue sealing plane.

More particularly, in order to effectively seal larger vessels (or tissue) two predominant mechanical parameters are accurately controlled—the pressure applied to the vessel (tissue) and the gap distance between the electrodes—both of which are affected by the thickness of the sealed vessel. More particularly, accurate application of pressure is important to oppose the walls of the vessel; to reduce the tissue impedance to a low enough value that allows enough electrosurgical energy through the tissue; to overcome the forces of expansion during tissue heating; and to contribute to the end tissue thickness which is an indication of a good seal. It has been determined that a typical fused vessel wall is optimum between about 0.001 and about 0.006 inches. Below this range, the seal may shred or tear and above this range the lumens may not be properly or effectively sealed.

With respect to smaller vessels, the pressure applied to the tissue tends to become less relevant whereas the gap distance between the electrically conductive surfaces becomes more significant for effective sealing. In other words, the chances of the two electrically conductive surfaces touching during activation increases as vessels become smaller.

It has been found that the pressure range for assuring a consistent and effective seal is between about 3 kg/cm² to about 16 kg/cm² and, desirably, within a working range of 7 kg/cm² to 13 kg/cm². Manufacturing an instrument which is capable of providing a closure pressure within this working range has been shown to be effective for sealing arteries, tissues and other vascular bundles.

Various force-actuating assemblies have been developed in the past for providing the appropriate closure forces to effect vessel sealing. For example, commonly-owned U.S. patent application Ser. Nos. 10/460,926 and 11/513,979 disclose two different envisioned actuating assemblies developed by Valleylab, Inc. of Boulder, Colo., a division of Tyco Healthcare LP (now Covidien, LP), for use with Valleylab's vessel sealing and dividing instruments commonly sold under the trademark LIGASURE®. The contents of both of these applications are hereby incorporated by reference herein.

During use, one noted challenge for surgeons has been the inability to manipulate the end effector assembly of the vessel sealer to grasp tissue in multiple planes, i.e., off-axis, while generating the above-noted required forces to effect a reliable vessel seal. It would therefore be desirable to develop an endoscopic or endoluminal vessel sealing instrument which includes an end effector assembly capable of being manipulated along multiple axes to enable the surgeon to grasp and seal vessels lying along different planes within a surgical cavity.

Endoluminal procedures often require accessing tissue deep in tortuous anatomy of a natural lumen using a flexible catheter or endoscope. Conventional vessel sealing devices may not be appropriate for use in some endoluminal procedures because of a rigid shaft that can not easily negotiate the tortuous anatomy of a natural lumen It would therefore be desirable to develop an endoscopic or endoluminal vessel sealing instrument having a flexible shaft capable of insertion in a flexible endoscope or catheter. In some instances, it may also be desirable to have the flexible shaft tend to maintain a straight or un-articulated configuration throughout the insertion into the flexible endoscope or catheter.

In other instances where a tensile load is applied to open and close the jaw members, or to articulate the end effector assembly, the flexible shaft may be compressed. This compression may result in unintentional movement in the instrument that may frustrate the intent of a surgeon. It would therefore be desirable to develop an endoscopic or endoluminal vessel sealing instrument having a flexible shaft exhibiting a suitable flexural rigidity to facilitate insertion in a flexible endoscope or catheter, and exhibiting a suitable axial rigidity to maintain an orientation of the flexible shaft during use of the instrument.

### SUMMARY

The present disclosure relates to an endoscopic surgical instrument for sealing tissue. The instrument includes an end effector having a pair of jaw members adapted to connect to a source of electrosurgical energy. At least one jaw member of the pair of jaw members is movable relative to the other to move the end effector between an open configuration wherein the jaw members are substantially spaced for receiving tissue and a closed configuration wherein the jaw members are closer together for contacting the tissue. A handle is provided being manually movable to selectively induce motion in the end effector between the open configuration and the closed configuration. An elongated shaft defines a longitudinal axis and includes distal and proximal ends. The distal end is coupled to the end effector and the proximal end is coupled to the handle. The elongated shaft includes a plurality of links arranged sequentially such that neighboring links engage one another across a pair of rotational edges defined by each of the links to maintain the end effector in an aligned configuration with respect to the longitudinal axis. Each of the rotational edges is substantially spaced in a lateral direction from the longitudinal axis and the neighboring links may pivot about the rotational edges to move the end effector to an articulated configuration.

The instrument may further include a pair of substantially elastic steering cables extending through at least one longitudinal cavity defined in the elongated shaft. The pair of steering cables may be coupled to a distal portion of the elongated shaft such that a differential tension in the pair of steering cables induces pivotal motion about the rotational edges to articulate the end effector in a first plane of articulation. A general tension may be imparted to the pair of steering cables when the end effector is in the aligned configuration.

The pair of rotational edges defined by one of the links may be radially offset from the pair of rotational edges defined by another of the plurality of links by about 90° to define a second plane of articulation that is substantially orthogonal to the first plane of articulation. The instrument may include a second pair of steering cables extending through the least one longitudinal cavity. The second pair of steering cables may be coupled to a distal portion of the elongated shaft such that a differential tension in the second pair of steering cables induces pivotal motion about the rotational edges to articulate the end effector in the second plane of articulation.

A substantially flat mating surface may extend between the pair of rotational edges, and the rotational edges may be rounded. At least one of the plurality of links may include a rib extending therefrom to engage a neighboring link and thereby discourage radial displacement between the neighboring links.

According to another aspect of the disclosure, an endoscopic surgical instrument for sealing tissue includes an end effector having a pair of jaw members adapted to connect to a source of electrosurgical energy. One or both jaw members is movable relative to the other to move the end effector between an open configuration wherein the jaw members are substantially spaced for receiving tissue and a closed configuration wherein the jaw members are closer together for contacting tissue. A handle is manually movable to selectively induce motion in the end effector between the open configuration and the closed configuration. An elongated shaft defines a longitudinal axis and includes distal and proximal ends. The distal end is coupled to the end effector and the proximal end is coupled to the handle. The elongated shaft includes a flexible portion to permit the end effector to articulate, and the flexible portion includes a plurality of links arranged sequentially such that neighboring links engage one another across substantially flat forward and trailing mating faces to maintain the end effector in an aligned configuration with respect to the longitudinal axis. One or both of the forward and trailing mating faces define a rotational edge thereof about which the neighboring links may pivot to move the end effector to an articulated configuration. At least one longitudinal cavity extends through the flexible portion, and at least one steering cable extends through the at least one longitudinal cavity. The steering cable is arranged to impart a compressive force on the plurality of links to maintain engagement between the mating faces.

One of the forward and trailing mating faces may define a first pair of rotational edges on opposing sides of the longitudinal axis such that the end effector articulates in opposite directions in a first plane of articulation upon pivoting of the neighboring links about each of the first pair rotational edges. One or more links of the plurality of links may define a second pair of rotational edges, the second pair of rotational edges oriented such that the end effector articulates in a second plane of articulation upon pivoting of neighboring links about the second first pair rotational edges. The second plane of articulation may be substantially orthogonal to the first plane of articulation.

In one embodiment, one or more the at least one steering cables may include a first pair of steering cables coupled to a distal end of the elongated shaft such that relative longitudinal movement between the first pair of steering cables induces articulation of the end effector in the first plane of articulation. The steering cables may further include a second pair of steering cables coupled to a distal end of the elongated shaft such that relative longitudinal motion between the second pair of steering cables induces articulation of the end effector in the second plane of articulation. Each link of the plurality of links may be similar in construction and each link may be oriented with a 90° offset with respect to neighboring links to orient the pair of rotational edges.

One or more of the links may include a rib extending therefrom to engage a neighboring link and thereby discourage radial displacement between the neighboring links. The steering cables may be substantially elastic.

According to another aspect of the disclosure, an endoscopic surgical instrument for sealing tissue includes an end effector having a pair of jaw members adapted to connect to a source of electrosurgical energy. At least one jaw member of the pair of jaw members is movable relative to the other to move the end effector between an open configuration wherein the jaw members are substantially spaced for receiving tissue and a closed configuration wherein the jaw members are closer together for contacting the tissue. A handle is provided being manually movable to selectively induce motion in the end effector between the open configuration and the closed configuration. An elongated shaft defines a longitudinal axis and includes distal and proximal ends. The distal end is coupled to the end effector and the proximal end is coupled to the handle. The elongated shaft includes a plurality of links arranged sequentially such that each of the links may pivot relative to a neighboring link to move the end effector between an aligned configuration and articulated configuration with respect to the longitudinal axis. Each of the links includes a substantially rigid base and a pair of relatively flexible tubes extending therefrom to engage the neighboring link.

The instrument may further include a pair of substantially elastic steering cables extending through at least one longitudinal cavity defined in the elongated shaft. The pair of steering cables may be coupled to a distal portion of the elongated shaft such that a differential tension in the pair of steering cables induces elastic bending in the pair of flexible tubes to articulate the end effector in a first plane of articulation. A general tension may be imparted to the pair of steering cables when the end effector is in the aligned configuration.

The pair of flexible tubes defined by one of the links may be radially offset from the pair of flexible tubes defined by another of the plurality of links by about 90° to define a second plane of articulation that is substantially orthogonal to the first plane of articulation. The instrument may include a second pair of steering cables extending through the least one longitudinal cavity. The second pair of steering cables may be coupled to a distal portion of the elongated shaft such that a differential tension in the second pair of steering cables induces bending of the flexible tubes to articulate the end effector in the second plane of articulation.

The longitudinal cavity may extend through the flexible tubes, and the flexible tubes may include a nitinol alloy. At least one of the plurality of links may include a rib extending therefrom to engage a neighboring link and thereby discourage radial displacement between the neighboring links.

According to another aspect of the disclosure, an endoscopic surgical instrument for sealing tissue includes an end effector having a pair of jaw members adapted to connect to a source of electrosurgical energy. One or both jaw members is movable relative to the other to move the end effector between an open configuration wherein the jaw members are substantially spaced for receiving tissue and a closed configuration wherein the jaw members are closer together for contacting tissue. A handle is manually movable to selectively induce motion in the end effector between the open configuration and the closed configuration. An elongated shaft defines a longitudinal axis and includes distal and proximal ends. The distal end is coupled to the end effector and the proximal end is coupled to the handle. The elongated shaft includes a flexible portion to permit the end effector to articulate, and the flexible portion includes a plurality of links. At least one of the links includes a substantially rigid base and at least one relatively flexible tube extending therefrom to engage the neighboring link maintain the end effector in an aligned configuration with respect to the longitudinal axis. A longitudinal cavity extends through the flexible tube, and at least one steering cable extends through the longitudinal cavity. The steering cable is arranged to impart a compressive force on the plurality of links to induce bending of the flexible tube to move the end effector to an articulated configuration.

A first pair of flexible tubes may be disposed on opposing sides of the longitudinal axis to define a first plane of articulation such that the end effector articulates in opposite directions in the first plane of articulation upon bending of the flexible tubes. One or more of the links may define a second pair of flexible tubes, the second pair of flexible tubes oriented such that the end effector articulates in a second plane of articulation upon bending of the flexible tubes. The second plane of articulation may be substantially orthogonal to the first plane of articulation.

In one embodiment, one or more the at least one steering cables may include a first pair of steering cables coupled to a distal end of the elongated shaft such that relative longitudinal movement between the first pair of steering cables induces articulation of the end effector in the first plane of articulation. The steering cables may further include a second pair of steering cables coupled to a distal end of the elongated shaft such that relative longitudinal motion between the second pair of steering cables induces articulation of the end effector in the second plane of articulation. Each link of the plurality of links may be similar in construction and each link may be oriented with a 90° offset with respect to neighboring links to orient the pair of flexible tubes.

One or more of the links may include a rib extending therefrom to engage a neighboring link and thereby discourage radial displacement between the neighboring links. The link may include a proximal rib projecting from the rigid base to engage a distal rib projecting from a rigid base of the neighboring link. The proximal rib may engage the distal rib across a substantially flat sliding face. The steering cables may be substantially elastic. One or more of the flexible tubes may include a nitinol alloy.

According to another aspect of the disclosure, an endoscopic surgical instrument for sealing tissue includes an end effector having a pair of jaw members adapted to connect to a source of electrosurgical energy. At least one of the jaw members is movable relative to the other to move the end effector between an open configuration wherein the jaw members are substantially spaced for receiving tissue and a closed configuration wherein the jaw members are closer together for contacting the tissue. A handle is manually movable to selectively induce motion in the end effector between the open configuration and the closed configuration. An elongated shaft defines a longitudinal axis and includes distal and proximal ends. The distal end is coupled to the end effector and the proximal end is coupled to the handle. The elongated shaft includes a flexible portion movable out of alignment with the longitudinal axis. The flexible portion exhibits a composite construction including an outer tubular layer defining a first wall thickness, and an inner tubular layer extending through the outer tubular layer and defining a second wall thickness. The inner tubular layer is relatively rigid with respect to the outer tubular layer, and the first wall thickness is relatively thick with respect to the second wall thickness.

The outer tubular layer may exhibit a modulus of elasticity of about 52,600 psi, and may include a thermoplastic elastomer. The inner tubular layer may exhibit a modulus of elasticity of about 6×10⁶ psi, and may include a nitinol tube. Alternatively, the inner tubular layer may include a stainless steel tube, and the stainless steel tube may include laterally extending notches formed therein to facilitate lateral bending of the flexible portion of the elongated shaft. The notches may be arranged in a helical pattern along a length of the tube.

The flexible portion of the elongated shaft may exhibit an axial rigidity of about 20,000 lb and flexural rigidity of about 60 lb·in². The second wall thickness may be about 9 percent of the first wall thickness. The flexible portion may exhibit sufficient axial rigidity to maintain a shape and orientation of the flexible portion in a non-aligned configuration with respect to the longitudinal axis during normal surgical use of the instrument. The flexible portion may include at least one passageway defined therein. The instrument may include one or more tensile members extending through the passageway and coupled to the end effector such that the tensile members are movable to induce motion in the end effector.

The elongated shaft may include an articulating portion movable between an aligned configuration and an articulated configuration with respect to the flexible portion. A pair of steering cables may be coupled to the end effector such that a differential tension in the pair of steering cables induces articulation of the end effector in a first plane of articulation. A general tension may be imparted to the pair of steering cables when the end effector is in the aligned configuration.

The articulating portion may include a plurality of links arranged sequentially such that each of the links may pivot relative to a neighboring link to move the articulating portion between the aligned and articulated configurations. A first pivoting axis defined by one of the links may be radially offset from a second pivoting axis defined by another of the plurality of links by about 90° such that a second plane of articulation is substantially orthogonal to the first plane of articulation. A second pair of steering cables may also extend through the passageway and may be coupled to the end effector such that a differential tension in the second pair of steering cables pivots the links about the second pivoting axis to induce articulation of the end effector in the second plane of articulation.

According to another aspect of the disclosure, an endoscopic surgical instrument for sealing tissue includes an end effector having a pair of jaw members adapted to connect to a source of electrosurgical energy. One or both jaw members is movable relative to the other to move the end effector between an open configuration wherein the jaw members are substantially spaced for receiving tissue and a closed configuration wherein the jaw members are closer together for contacting tissue. A handle is manually movable to selectively induce motion in the end effector between the open configuration and the closed configuration. An elongated shaft defines a longitudinal axis and includes distal and proximal ends. The distal end is coupled to the end effector and the proximal end is coupled to the handle. The elongated shaft includes a flexible portion to permit the end effector to articulate with respect to the longitudinal axis. The flexible portion includes an anisotropic tube exhibiting a modulus of elasticity that generally decreases as a function of radius.

According to another aspect of the disclosure, an endoscopic surgical instrument for sealing tissue includes an end effector having a pair of jaw members adapted to connect to a source of electrosurgical energy. At least one of the jaw members is movable relative to the other to move the end effector between an open configuration wherein the jaw members are substantially spaced for receiving tissue and a closed configuration wherein the jaw members are closer together for contacting the tissue. A handle is manually movable to selectively induce motion in the end effector between the open configuration and the closed configuration. An elongated shaft defines a longitudinal axis and includes distal and proximal ends. The distal end is coupled to the end effector and the proximal end is coupled to the handle. The elongated shaft includes a flexible portion movable out of alignment with the longitudinal axis. The flexible portion includes a helical passageway extending therethrough. A tensile member extending through the helical passageway is coupled to the end effector, such that the tensile member is movable to induce motion in the end effector.

The helical passageway may traverse a radial arc of about 360 degrees, and may be configured as a helical lumen extending through an interior of the flexible portion of the elongated shaft. The flexible portion of the elongated shaft may exhibit sufficient rigidity to maintain a shape and orientation of the flexible portion during normal surgical use of the instrument. The flexible portion may also include a composite of a flexible tube and a rigidizing element.

The elongated shaft may include an articulating portion movable between an aligned configuration and an articulated configuration with respect to the flexible portion. A pair of steering cables may be coupled to the end effector such that a differential tension in the pair of steering cables induces articulation of the end effector in a first plane of articulation. A general tension may be imparted to the pair of steering cables when the end effector is in the aligned configuration.

The articulating portion may include a plurality of links arranged sequentially such that each of the links may pivot relative to a neighboring link to move the articulating portion between the aligned and articulated configurations. A first pivoting axis defined by one of the links may be radially offset from a second pivoting axis defined by another of the plurality of links by about 90° such that a second plane of articulation is substantially orthogonal to the first plane of articulation. A second pair of steering cables may also extend through a helical passageway and may be coupled to the end effector such that a differential tension in the second pair of steering cables pivots the links about the second pivoting axis to induce articulation of the end effector in the second plane of articulation.

According to another aspect of the disclosure, an endoscopic surgical instrument for sealing tissue includes an end effector having a pair of jaw members adapted to connect to a source of electrosurgical energy. One or both jaw members is movable relative to the other to move the end effector between an open configuration wherein the jaw members are substantially spaced for receiving tissue and a closed configuration wherein the jaw members are closer together for contacting tissue. A handle is manually movable to selectively induce motion in the end effector between the open configuration and the closed configuration. An elongated shaft defines a longitudinal axis and includes distal and proximal ends. The distal end is coupled to the end effector and the proximal end is coupled to the handle. The elongated shaft includes a flexible portion to permit the end effector to articulate with respect to the longitudinal axis. A shaft axis extends centrally through the flexible portion. A passageway extending through the flexible portion includes a first longitudinal length disposed on a first lateral side of the shaft axis and a second longitudinal length disposed on an opposed lateral side of the shaft axis. A tensile member extends through the passageway and is coupled to the end effector such that longitudinal motion of the tensile member induces motion in the end effector.

The passageway may be helically arranged through the flexible portion and the first and second longitudinal lengths may be about equal with respect to one another. The passageway may be configured as a groove defined on an exterior surface of a tubular member.

The elongated shaft may include an articulating portion movable between an aligned configuration and an articulated configuration with respect to the longitudinal axis. Longitudinal motion of the tensile member may induce movement of the articulation portion between the aligned and articulated configurations. The tensile member may be substantially elastic and the articulating portion may include a plurality of links arranged sequentially such that each of the links may pivot relative to a neighboring link to move the articulating portion between the aligned and articulated configurations.

### BRIEF DESCRIPTION OF THE DRAWINGS

Various embodiments of the subject instrument are described herein with reference to the drawings wherein:

FIG. 1 is a perspective view of an endoscopic forceps depicting a housing, a flexible shaft, articulation assembly and an end effector assembly according to the present disclosure;

FIG. 2 is an enlarged, exploded perspective view of the end effector and flexible shaft of FIG. 1 depicting a plurality of links forming the flexible shaft;

FIG. 3 is an enlarged, perspective view of a link of FIG. 2 depicting a forward male face of the link;

FIG. 4 is an enlarged, perspective view of a neighboring link of FIG. 2 depicting a trailing female face of the neighboring link;

FIG. 5 is an enlarged, perspective view of an underside of the articulation assembly of FIG. 1;

FIG. 6 is an exploded, perspective view of the articulation assembly;

FIG. 7 is a bottom view of the articulation assembly in a “home” configuration for maintaining the flexible shaft in a non-articulated orientation;

FIG. 8 is an enlarged, top view of the flexible shaft in the non-articulated orientation corresponding to the “home” configuration of the articulation assembly;

FIG. 9 is a bottom view of the articulation assembly in a configuration corresponding to a RIGHT articulated orientation of the flexible shaft;

FIG. 10 is a bottom view of the articulation assembly in a configuration corresponding to a LEFT articulated orientation of the flexible shaft;

FIG. 11 is an enlarged, top view of the flexible shaft in the RIGHT articulated orientation;

FIG. 12 is an enlarged, side view of a distal end of the flexible shaft in the non-articulated orientation;

FIG. 13 is an enlarged, side view of the flexible shaft in an UP articulated orientation;

FIG. 14 is an enlarged, exploded perspective view of the end effector of FIG. 2 and an alternate embodiment of a flexible shaft depicting a plurality of links of an alternate configuration forming the flexible shaft;

FIG. 15 is an enlarged, perspective view of a link of FIG. 14;

FIG. 16 is an enlarged, perspective view of a plurality of links of FIG. 15 assembled for articulation with respect to neighboring links in orthogonal directions;

FIG. 17 is a bottom view of the articulation assembly in the “home” configuration of FIG. 7 for maintaining the flexible shaft of FIG. 14 in a non-articulated orientation;

FIG. 18 is an enlarged, top view of the flexible shaft of FIG. 14 in the non-articulated orientation corresponding to the “home” configuration of the articulation assembly;

FIG. 19 is an enlarged, top view of the flexible shaft of FIG. 14 in a RIGHT articulated orientation;

FIG. 20 is an enlarged, side view of a distal end of the flexible shaft of FIG. 14 in the non-articulated orientation;

FIG. 21 is an enlarged, side view of the flexible shaft of FIG. 14 in an UP articulated orientation;

FIG. 22 is an enlarged, perspective view of a plurality of links of another alternate embodiment assembled for articulation with respect to neighboring links in orthogonal directions;

FIG. 23 is an enlarged, exploded perspective view of the end effector of FIG. 2 and yet another alternate embodiment of a flexible shaft depicting a plurality of links of an alternate configuration forming an articulating portion of the elongated shaft, and a flexible tube forming a flexible portion of the elongated shaft;

FIG. 24 is an enlarged, cross-sectional view of the flexible tube of FIG. 23 depicting a composite construction;

FIG. 25 is a cross-sectional view of an alternate embodiment of a flexible tube depicting a uniform construction;

FIG. 26 is a cross-sectional view of the flexible tube of FIG. 25 encircling a guide tube;

FIG. 27 is a front view of a tubular member for constructing an alternate embodiment of a flexible tube with a composite construction;

FIG. 28 is a bottom view of the articulation assembly of FIG. 1 in the “home” configuration of FIG. 7 for maintaining the flexible shaft of FIG. 23 in a non-articulated orientation;

FIG. 29 is an enlarged, top view of the elongated shaft of FIG. 23 wherein the articulating portion is in the non-articulated orientation corresponding to the “home” configuration of the articulation assembly and the flexible portion is in an aligned configuration;

FIG. 30 is a top view of the elongated shaft of FIG. 23 wherein the articulating portion is in the non-articulated orientation and the flexible portion having a composite construction is in a non-aligned orientation;

FIG. 31 is a top view of an alternate embodiment of an elongated shaft wherein an articulating portion is in an articulated orientation and a flexible portion having a uniform construction is in a non-aligned orientation;

FIG. 32 is a bottom view of the articulation assembly in a configuration corresponding to a RIGHT articulated orientation of the articulating portion of the elongated shaft of FIG. 23;

FIG. 33 is a top view of the elongated shaft of FIG. 23, wherein the articulating portion is in the RIGHT articulated orientation;

FIG. 34 is a bottom view of the articulation assembly in a configuration corresponding to a LEFT articulated orientation of the articulating portion of the elongated shaft of FIG. 23;

FIG. 35 is an enlarged, top view of the elongated shaft of FIG. 23, wherein the articulating portion is in the LEFT articulated orientation;

FIG. 36 is an enlarged, side view of a distal end of the elongated shaft of FIG. 23, wherein the articulating portion is in the non-articulated orientation;

FIG. 37 is an enlarged, side view of the elongated shaft of FIG. 23, wherein the articulating portion is in an UP articulated orientation;

FIG. 38 is an enlarged, exploded perspective view of the end effector of FIG. 2 and yet another alternate embodiment of a flexible shaft depicting the plurality of links of FIG. 23 forming an articulating portion of the elongated shaft, and a flexible tube of an alternate construction forming a flexible portion of the elongated shaft;

FIG. 39 is an enlarged, perspective view of the flexible tube of FIG. 38 depicting interior helical lumens;

FIG. 40 is a perspective view of an alternate embodiment of a flexible tube depicting exterior helical grooves;

FIG. 41 is a bottom view of the articulation assembly in a “home” configuration for maintaining the articulating portion of the elongated shaft of FIG. 38 in a non-articulated orientation;

FIG. 42 is an enlarged, top view of the elongated shaft of FIG. 38 wherein the articulating portion is in the non-articulated orientation corresponding to the “home” configuration of the articulation assembly and the flexible portion is in an aligned configuration;

FIG. 43 is a top view of the elongated shaft wherein the articulating portion of the elongated shaft of FIG. 38 is in the non-articulated orientation and the flexible portion having helical lumens is in a non-aligned orientation;

FIG. 44 is a top view of an alternate embodiment of an elongated shaft wherein a flexible portion having axial lumens is in a non-aligned orientation;

FIG. 45 is a bottom view of the articulation assembly in a configuration corresponding to a RIGHT articulated orientation of the articulating portion of the elongated shaft of FIG. 38;

FIG. 46 is a top view of the elongated shaft of FIG. 41, wherein the articulating portion is in the RIGHT articulated orientation;

FIG. 47 is a bottom view of the articulation assembly in a configuration corresponding to a LEFT articulated orientation of the articulating portion of the articulating portion of the elongated shaft of FIG. 38; and

FIG. 48 is an enlarged, top view of the elongated shaft of FIG. 38, wherein the articulating portion is in the LEFT articulated orientation.

### DETAILED DESCRIPTION

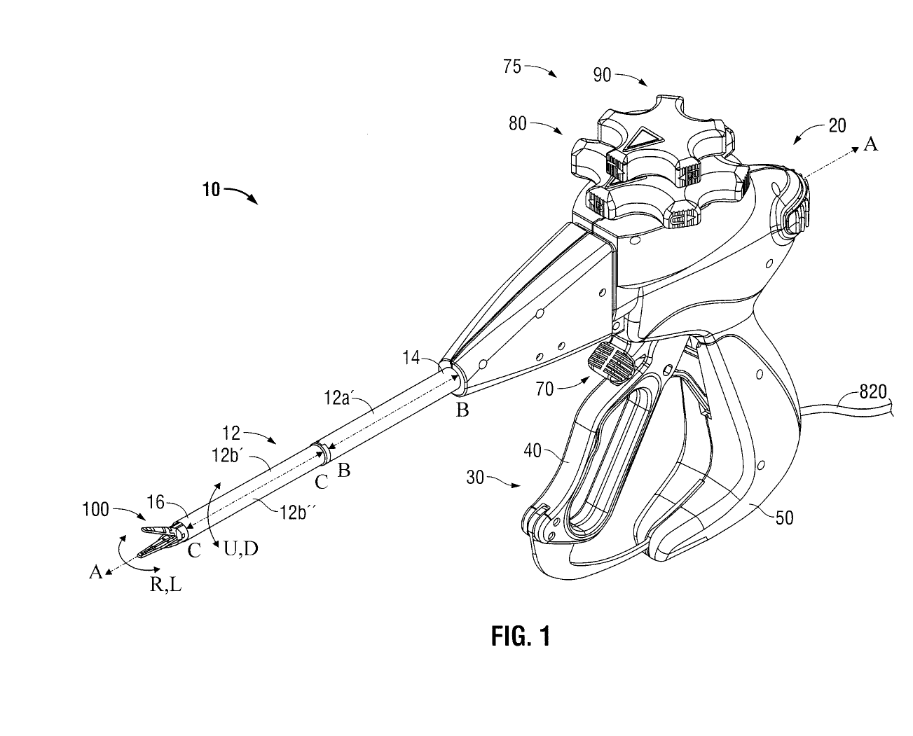

Referring initially to FIG. 1, one embodiment of an endoscopic vessel sealing forceps is depicted generally as 10. In the drawings and in the descriptions which follow, the term “proximal,” as is traditional, will refer to the end of the forceps 10 which is closer to the user, while the term “distal” will refer to the end which is farther from the user. The forceps 10 comprises a housing 20, an end effector assembly 100 and an elongated shaft 12 extending therebetween to define a longitudinal axis A-A. A handle assembly 30, an articulation assembly 75 composed of two articulation controls 80 and 90 and a trigger assembly 70 are operable to control the end effector assembly 100 to effectively grasp, seal and divide tubular vessels and vascular tissue. Although the forceps 10 is configured for use in connection with bipolar surgical procedures, various aspects of the present disclosure may also be employed for monopolar surgical procedures.

Forceps 10 includes an electrosurgical cable 820, which connects the forceps 10 to a source of electrosurgical energy, e.g., a generator (not shown). It is contemplated that generators such as those sold by Covidien—Energy-based Devices, located in Boulder, Colo. may be used as a source of electrosurgical energy, e.g., Covidien's LIGASURE™ Vessel Sealing Generator and Covidien's Force Triad™ Generator. Cable 820 may be internally divided into numerous leads (not shown), which each transmit electrosurgical energy through respective feed paths through the forceps 10 for connection to the end effector assembly 100.

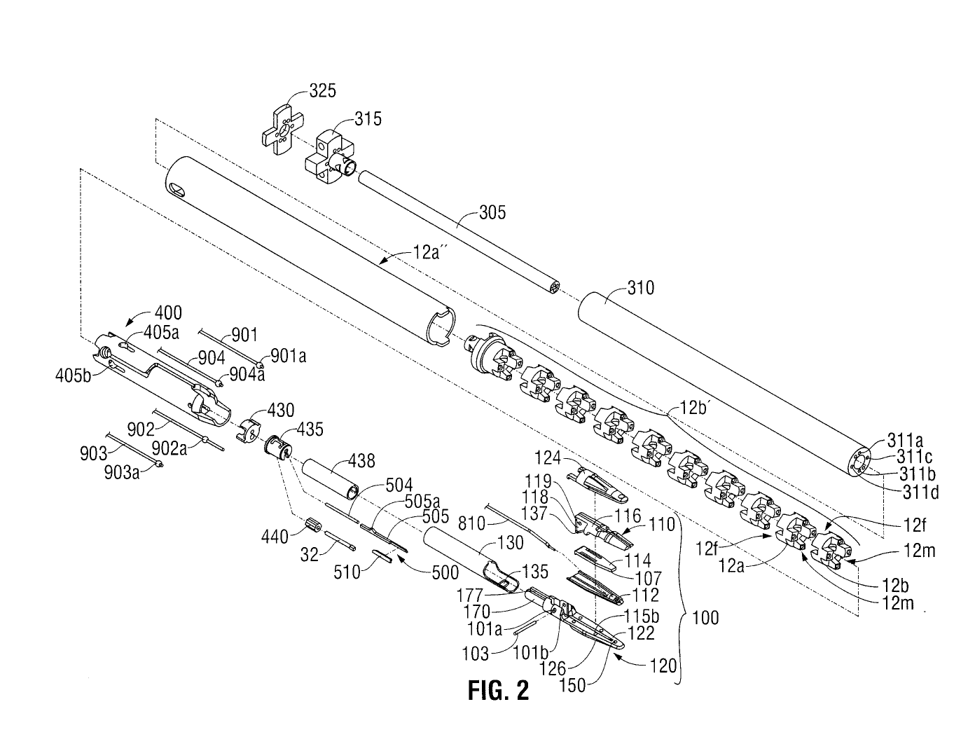

Handle assembly 30 includes a fixed handle 50 and a movable handle 40. The fixed handle 50 is integrally associated with the housing 20, and the movable handle 40 is movable relative to fixed handle 50 to induce relative movement between a pair of jaw members 110, 120 (FIG. 2) of the end effector assembly 100. The movable handle 40 is operatively coupled to the end effector assembly 100 via a drive rod 32 (see FIG. 2), which extends through the elongated shaft 12, and reciprocates to induce movement in the jaw members 110, 120. The movable handle 40 may be approximated with fixed handle 50 to move the jaw members 110 and 120 from an open position wherein the jaw members 110 and 120 are disposed in spaced relation relative to one another, to a clamping or closed position wherein the jaw members 110 and 120 cooperate to grasp tissue therebetween. Electrosurgical energy may be transmitted through tissue grasped between jaw members 110, 120 to effect a tissue seal.

Trigger assembly 70 is operable to advance a blade 510 (FIG. 2) through a knife channel, e.g., 115b defined in the jaw members 110, 120 to transect sealed tissue. The trigger assembly 70 is operatively coupled to the blade 510 via a knife rod 504 (FIG. 2), which extends through the elongated shaft 12. Various aspects of the end effector assembly 100, the housing 20, handle assembly 30, the trigger assembly 70 and the operation of these mechanisms to electrosurgically treat tissue are discussed in greater detail in commonly owned U.S. Provisional Application No. 61/157,722, the entire content of which is incorporated by reference herein.

Elongated shaft 12 defines a distal end 16 dimensioned to mechanically engage the end effector assembly 100 and a proximal end 14, which mechanically engages the housing 20. The elongated shaft 12 includes two distinct portions, a proximal portion 12a′ defining a proximal shaft axis B-B and a distal portion 12b′ defining a distal shaft axis C-C.

The proximal portion 12a′ of the shaft 12 may exhibit various constructions. For example, the proximal portion 12a′ may be formed from a substantially rigid tube, from flexible tubing (e.g., plastic), or the proximal portion 12a′ may be formed as a composite of a flexible tube and a rigidizing element, such as a tube of braided steel, to provide axial (e.g., compressional) and rotational strength. In other embodiments, the proximal portion 12a′ may be constructed from a plastically deformable material.

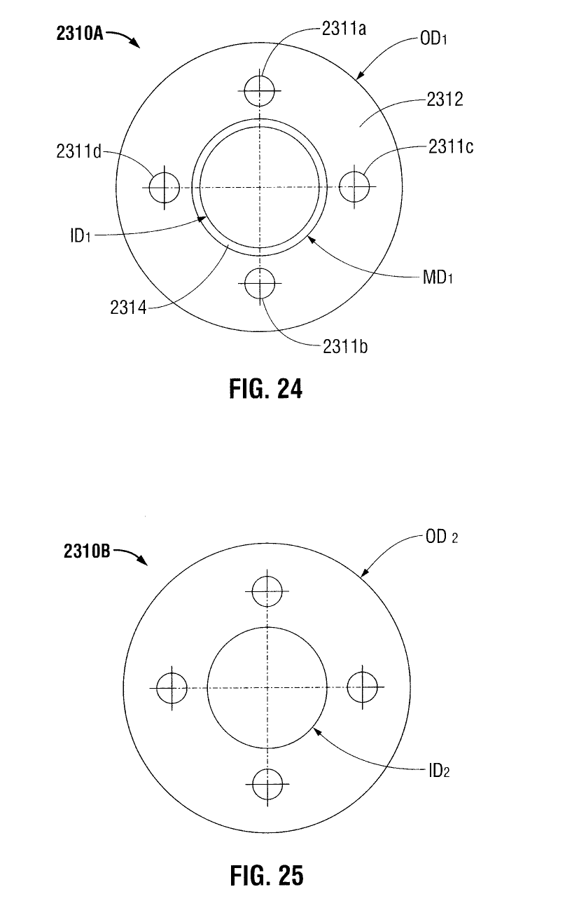

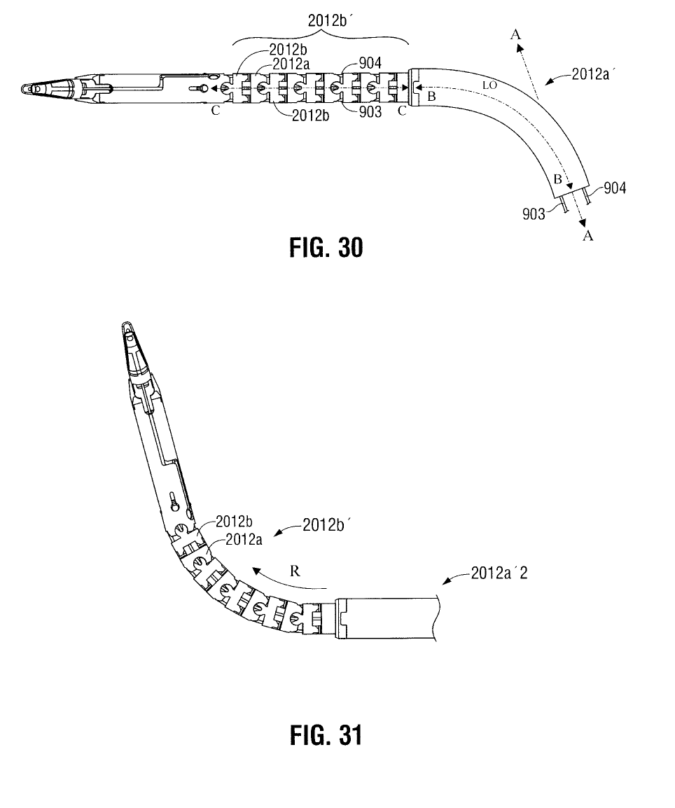

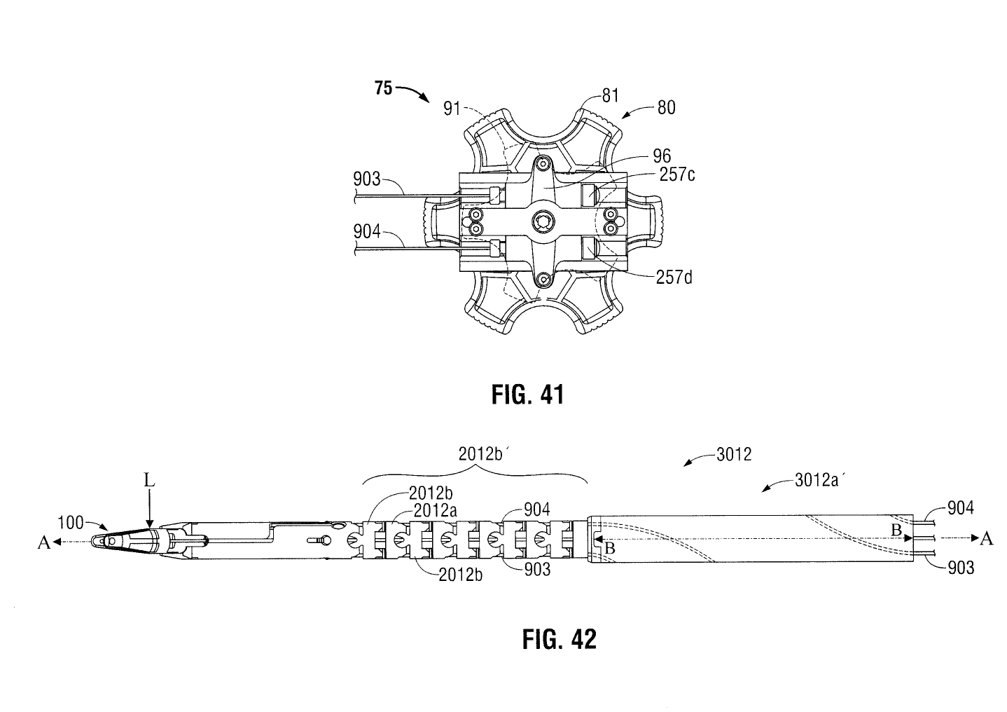

In an embodiment as described below with reference to FIGS. 30, 33 and 35, a proximal portion 2012a′ exhibits a flexural rigidity that is sufficiently low to permit a surgeon to pre-shape or reshape the proximal portion 12a′ prior to or during a surgical procedure to accommodate the contours and characteristics of the surgical site. Once shaped, the proximal end portion 2012a′ may define a non-aligned configuration wherein the proximal shaft axis B-B is substantially out of alignment with the longitudinal axis A-A. The proximal portion 2012a′ also exhibits an axial rigidity that is sufficient to maintain the shape and orientation of the non-aligned configuration during normal surgical use. As described with reference to FIG. 24 below, a composite structure of the proximal portion 2012a′ permits an appropriate balance to be maintained between the flexural and axial rigidity. In another embodiment as described below with reference to FIGS. 41, 44 and 46, a proximal portion 3012a′ permits a surgeon to pre-shape or reshape the proximal portion 3012a′. As described with reference to 39, a component of the proximal portion 3012a′ includes helical lumens that permit the proximal portion 3012a′ to maintain the shape and orientation of the non-aligned configuration during normal surgical use.

The distal portion 12b′ of shaft 12 includes an exterior casing or insulating material 12b″ disposed over a plurality of links 12a, 12b (see FIG. 2). The links 12a and 12b are configured to pivot relative to one another to permit the distal portion 12b′ of the shaft 12 to articulate relative to the proximal shaft axis B-B. In one embodiment, the links 12a and 12b are nestingly engaged with one another to pelf lit pivotal motion of the distal portion 12b′ in two orthogonal planes in response to movement of articulation controls 80 and 90. The links 12a and 12b may be shaped to permit the distal portion 12b′ of the shaft 12 to be self-centering, or to have a tendency to return to an unarticulated configuration. As described below with reference to FIG. 16, for example, self centering links 1012a and 1012b may exhibit alternate configurations.

Articulation assembly 75 sits atop housing 20 and is operable via articulation controls 80 and 90 to move the end effector assembly 100 (and the articulating distal portion 12b′of the shaft 12) in the direction of arrows “U, D” and “R, L” relative to axis proximal shaft axis B-B as explained in more detail below. Controls 80 and 90 may be provided in alternative arrangements such as disposed on the side of housing 20. Also, controls 80 and 90 may be replaced by other mechanisms to articulate the end effector 100 such as levers, trackballs, joysticks, or the like.

Referring now to FIG. 2, the flexible portion 12b′ of shaft 12 includes a plurality of links 12a and 12b. Each link 12a pivotally engages a neighboring link 12b to permit the flexible portion 12b′ of the shaft 12 to articulate the end effector assembly 100. Links 12a are similar in construction to links 12b in that each link 12a, 12b exhibits a forward male face 12m and a trailing female face 12f on an opposite side of the link Links 12a, and 12b exhibit a geometry that permits a male face 12m of a link 12a to nest within the female face 12f of neighboring link 12b when the link 12a is oriented with a ninety degree (90°) radial offset with respect to the neighboring link 12b. Such an alternating orientation of the links 12a, 12b facilitates articulation of the end effector 100 in orthogonal planes.

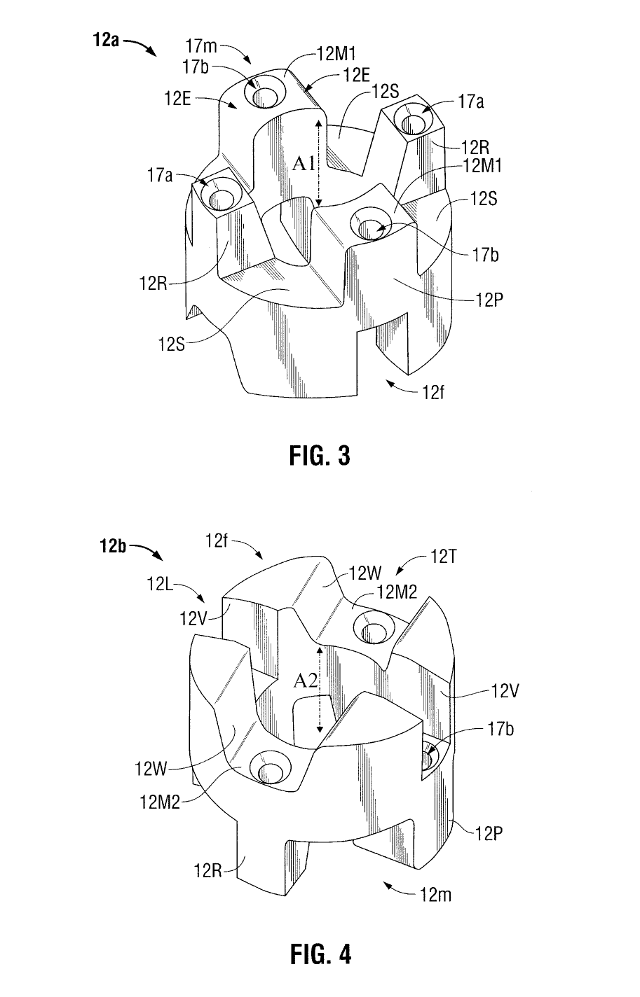

Referring to FIG. 3, the male face 12m of the link 12a includes a pair of pivots 12P and a pair of ribs 12R extending longitudinally from a proximal surface 12S thereof. The pivots 12P each include a substantially flat forward mating face 12M1 lying in a plane that is generally orthogonal to a longitudinal axis A1 defined by the link 12a. Two lateral edges 12E of the forward mating face 12M1 define rotational edges about which the link 12a can rotate with respect to a neighboring link 12b. The two rotational edges 12E are generally parallel with one another and are substantially spaced from the longitudinal axis A1 in opposing lateral directions. The edges 12E are also rounded to facilitate rotation of the links 12a, 12b thereabout.

Referring to FIG. 4, the female face 12f of link 12b includes a trough 12T extending therethrough in a lateral direction and a lateral slot 12L extending orthogonally to the trough 12T. The trough 12T receives the pair of pivots 12P of a neighboring link 12a, and includes a substantially flat mating face 12M2 to engage the forward mating faces 12M1 of the link 12a. The mating face 12M2 lies in a plane that is generally orthogonal to a longitudinal axis A2 defined by the link 12b. Thus, when the mating face 12M2 of a link 12b engages the forward mating face 12M1 of a link 12a, the axes A1 and A2 may be substantially aligned. The trough 12T exhibits angled walls 12W providing clearance for the link 12a to pivot within the trough 12T. The longitudinal slot 12L exhibits vertical walls 12V and receives the ribs 12R of a neighboring link 12a therein. The walls 12V of the slot 12L engage the ribs 12R to discourage radial displacement between neighboring links 12a, 12b.

The links 12a, 12b each include a central lumen 19a extending longitudinally theretrhrough. The central lumens 19a permits passage of various actuators, e.g., drive rod 32 and knife rod 504, and other components through the elongated shaft 12. Links 12a and 12b also define two pairs of opposed lumens 17a and 17b formed radially outward from the central lumen 19a. Each of the lumens 17a and 17b on a link 12a is radially spaced at a 90° from the neighboring lumen 17a, 17b such that each lumen 17a aligns with a lumen 17b of a neighboring link 12b. The lumens 17a and 17b cooperate to define a longitudinal cavity to permit passage of four steering cables 901, 902, 903 and 904 (FIG. 2) through the elongated shaft 12.

Referring again to FIG. 2, a link support 320 includes a mating face similar to the male face 12m of a link 12a to interface with a trailing link 12b. A proximal end of the link support 320 is fixedly mounted to an outer casing 12a″, which extends over the proximal portion 12a′ of the elongated shaft 12. An end effector support 400 includes a mating face similar to the female face 12f of a link 12b to interface with a leading link 12a.

The four steering cables 901-904 may be substantially elastic and slideably extend through lumens pairs 17a, and 17b defined in the links 12a and 12b. A distal end of the each of the steering cables 901-904 is coupled to an end effector support 400. More particularly, each steering cable 901-904 includes a ball-like mechanical interface at the distal end, namely, interfaces 901a-904a. Each interface 901a-904a is configured to securely mate within a corresponding recess defined in the end effector support 400. Interface 904a engages recess 405a, interface 903a engages recess 405b, and interfaces 901a and 902a engage similar recess on the end effector support 400

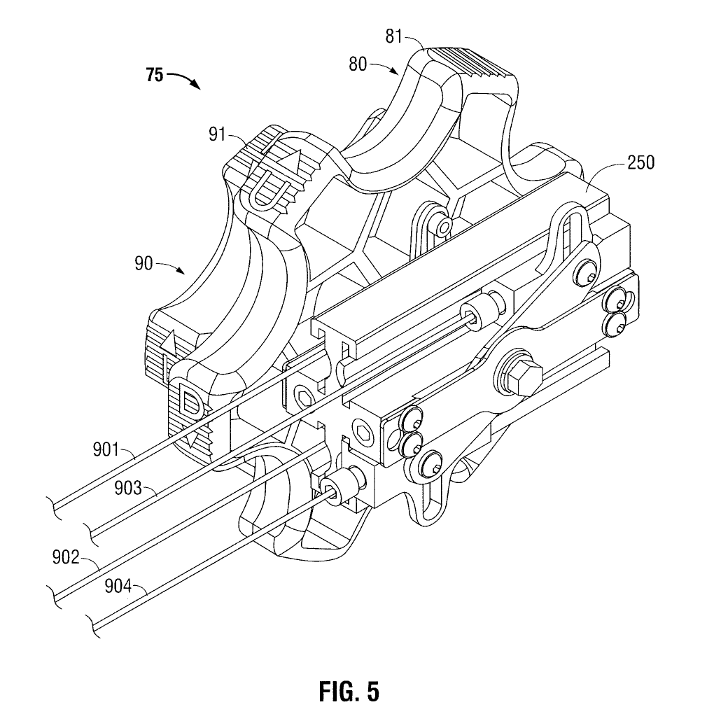

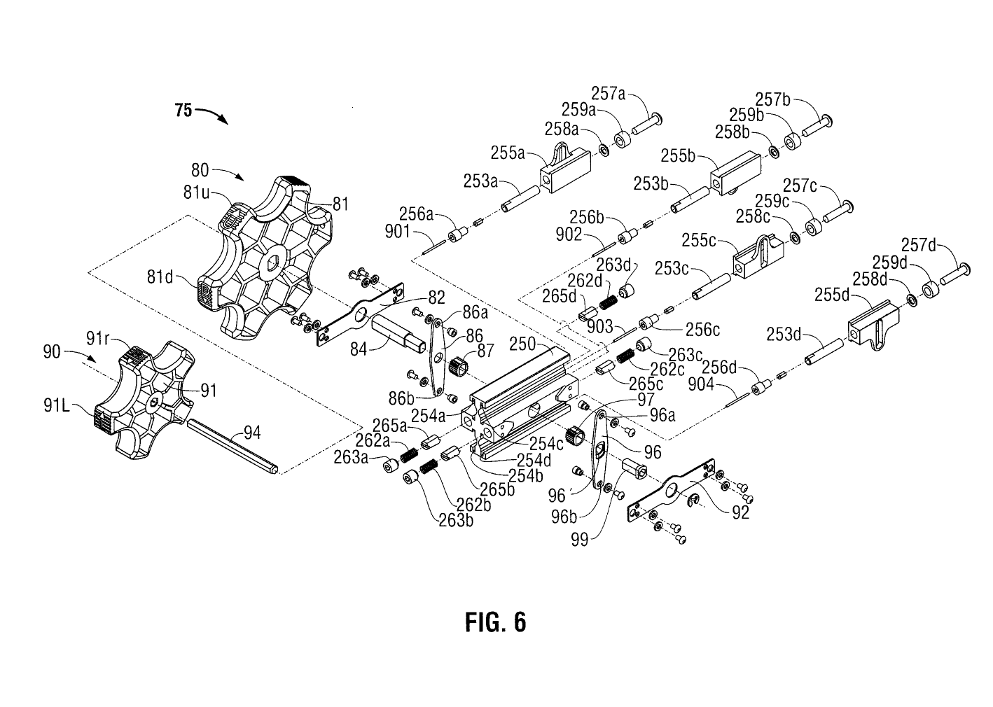

Proximal ends of the steering cables 901-904 are operatively coupled to the articulation controls 80, 90 as described below with reference to FIGS. 5 and 6. The steering cables 901-904 extend through the shaft 12 through a series of passageways defined therein. More particularly, a cross-shaped cable guide adapter 315 and guide adapter liner or washer 325 include bores defined therethrough to initially orient the cables 901-904 for passage through an outer tube 310 at 90° degree angles relative to one another. The adapter 315 also facilitates attachment of the shaft 12 to the housing 20. The tube 310 includes passageways 311a-311d defined therein to orient the cables 901-904, respectively, for reception into the lumens 17a and 17b (see FIGS. 3 and 4) of links 12a and 12b for ultimate connection to the end effector support 400 as described above.

A central guide tube 305 is utilized to orient the drive rod 32 and the knife rod 504 through the shaft 12 for ultimate connection to jaw member 110 and a knife assembly 500. The central guide tube 305 also guides an electrical lead 810 for providing electrosurgical energy to the jaw member 110. The central guide tube 305 is dimensioned for reception within outer tube 310, and may extend distally therefrom into the central lumens 19a defined in the links 12a and 12b. One or more steering cables, e.g., 902, includes a distal portion 902b that electrically connects to the end effector support 400 which, in turn, connects to jaw member 120. A return path (i.e., ground path) may thus be established through tissue captured between jaw members 110 and 120 for electrosurgical energy provided through jaw member 110.

The central extrusion or guide tube 305 is constructed from a highly flexible and lubricious material and performs several important functions: tube 305 guides the drive rod 32, the knife rod 504 and the electrical lead 810 from the guide adapter 315, shaft 12 and flexible shaft 12b′ to the end effector support 400 and knife assembly 500; the tube 305 provides electrical insulation between component parts; the tube 305 keeps the lead 810 and rods 32 and 504 separated during relative movement thereof; the tube 305 minimizes friction and clamping force loss; and tube 305 keeps the lead 810 and rods 32 and 504 close to the central longitudinal axis to minimize stretching during articulation. The tube 305 (and internal lumens) may be made from or include materials like polytetrafluoroethene (PTFE), graphite or other lubricating agents to minimize friction and other common losses associated with relative movement of component parts. Alternatively, a coaxial structure (not shown) may be utilized to guide the drive rod 32 and knife rod 504.

One or more distal guide plates 430 and an adapter 435 may also be utilized to further align the drive rod 32 and knife rod 504 and facilitate actuation of the jaw members 110 and 120. More particularly, alignment of the drive rod 32 facilitates opening and closing the jaw members 110, 120. A sleeve 130 includes an aperture 135 to engage a flange 137 of jaw member 110 such that axial movement of the sleeve 130 forces jaw member 110 to rotate around pivot pin 103 and clamp tissue. Sleeve 130 connects to adapter 435 which secures drive rod 32 therein via a wire crimp 440. The drive rod 32 has a flat 32a at a distal end thereof to reinforce attachment to crimp 440. By actuating movable handle 40 (FIG. 1), the drive rod 32 retracts sleeve 130 to close jaw member 110 about tissue. Pulling the sleeve 130 proximally closes the jaw members 110 and 120 about tissue grasped therebetween and pushing the sleeve 130 distally opens the jaw members 110 and 120 for grasping purposes. The end effector assembly 100 is designed as a unilateral assembly, i.e., jaw member 120 is fixed relative to the shaft 12 and jaw member 110 pivots about a pivot pin 103 to grasp tissue.

Also, alignment of knife rod 504 facilitates longitudinal movement of blade 510. Knife channel 115b runs through the center of jaw member 120 and a similar knife channel (not shown) extends through the jaw member 110 such that the blade 510 can cut the tissue grasped between the jaw members 110 and 120 when the jaw members 110 and 120 are in the closed position.

Jaw member 110 also includes a jaw housing 116 which has an insulative substrate or insulator 114 and an electrically conducive surface 112. Housing 116 and insulator 114 are dimensioned to securely engage the electrically conductive sealing surface 112. This may be accomplished by stamping, by overmolding, by overmolding a stamped electrically conductive sealing plate and/or by overmolding a metal injection molded seal plate. For example, the electrically conductive sealing plate 112 may include a series of upwardly extending flanges that are designed to matingly engage the insulator 114. The insulator 114 includes a shoe-like interface 107 disposed at a distal end thereof which is dimensioned to engage the outer periphery of the housing 116 in a slip-fit manner. The shoe-like interface 107 may also be overmolded about the outer periphery of the jaw 110 during a manufacturing step. It is envisioned that lead 810 terminates within the shoe-like interface 107 at the point where lead 810 electrically connects to the seal plate 112 (not shown). The movable jaw member 110 also includes a wire channel (not shown) that is designed to guide electrical lead 810 into electrical continuity with sealing plate 112.

All of these manufacturing techniques produce jaw member 110 having an electrically conductive surface 112 which is substantially surrounded by an insulating substrate 114 and housing 116. The insulator 114, electrically conductive sealing surface 112 and the outer, jaw housing 116 are dimensioned to limit and/or reduce many of the known undesirable effects related to tissue sealing, e.g., flashover, thermal spread and stray current dissipation. Alternatively, it is also envisioned that jaw members 110 and 120 may be manufactured from a ceramic-like material and the electrically conductive surface(s) 112 are coated onto the ceramic-like jaw members 110 and 120.

Jaw member 110 also includes a pivot flange 118 which includes the protrusion 137. Protrusion 137 extends from pivot flange 118 and includes an arcuately-shaped inner surface dimensioned to matingly engage the aperture 135 of sleeve 130 upon retraction thereof. Pivot flange 118 also includes a pin slot 119 that is dimensioned to engage pivot pin 103 to allow jaw member 110 to rotate relative to jaw member 120 upon retraction of the reciprocating sleeve 130. Pivot pin 103 also mounts to the stationary jaw member 120 through a pair of apertures 101a and 101b disposed within a proximal portion of the jaw member 120.

Jaw member 120 includes similar elements to jaw member 110 such as jaw housing 126 and an electrically conductive sealing surface 122. Likewise, the electrically conductive surface 122 and the insulative housing 126, when assembled, define the longitudinally-oriented channel 115a for reciprocation of the knife blade 510. As mentioned above, when the jaw members 110 and 120 are closed about tissue, the knife channel 115b permits longitudinal extension of the blade 510 to sever tissue along the tissue seal.

Jaw member 120 includes a series of stop members 150 disposed on the inner facing surfaces of the electrically conductive sealing surface 122 to facilitate gripping and manipulation of tissue and to define a gap “G” of about 0.001 inches to about 0.006 inches between opposing jaw members 110 and 120 during sealing and cutting of tissue. It is envisioned that the series of stop members 150 may be employed on one or both jaw members 110 and 120 depending upon a particular purpose or to achieve a desired result. A detailed discussion of these and other envisioned stop members 150 as well as various manufacturing and assembling processes for attaching and/or affixing the stop members 150 to the electrically conductive sealing surfaces 112, 122 are described in commonly-assigned, U.S. Pat. No. 7,473,253 entitled “VESSEL SEALER AND DIVIDER WITH NON-CONDUCTIVE STOP MEMBERS” by Dycus et al. which is hereby incorporated by reference in its entirety herein.

Jaw member 120 is designed to be fixed to the end of a tube 438, which is part of the distal articulating portion 12b′ of the shaft 12. Thus, articulation of the distal portion 12b′ of the shaft 12 will articulate the end effector assembly 100. Jaw member 120 includes a rear C-shaped cuff 170 having a slot 177 defined therein that is dimensioned to receive a slide pin 171 disposed on an inner periphery of tube 438. More particularly, slide pin 171 extends substantially the length tube 438 to slide into engagement (e.g., friction-fit, glued, welded, etc) within slot 177. C-shaped cuff 170 inwardly compresses to assure friction-fit engagement when received within tube 438. Tube 438 also includes an inner cavity defined therethrough that reciprocates the knife assembly 500 upon distal activation thereof. The knife blade 510 is supported atop a knife support 505. The knife rod 504 feeds through adapter 435 and operably engages a butt end 505a of the knife support 505. By actuating trigger assembly 70, the knife rod 504 is forced distally into the butt end 505a which, in turn, forces the blade 510 through tissue held between the jaw members 110 and 120. The knife rod 504 may be constructed from steel or other hardened substances to enhance the rigidity of the rod along the length thereof.

As mentioned above, the jaw members 110 and 120 may be opened, closed and articulated to manipulate tissue until sealing is desired. This enables the user to position and re-position the forceps 10 (FIG. 1) prior to activation and sealing. The unique feed path of the electrical lead 810 through the housing, along shaft 12 and, ultimately, to the jaw member 110 enables the user to articulate the end effector assembly 100 in multiple directions without tangling or causing undue strain on electrical lead 810.

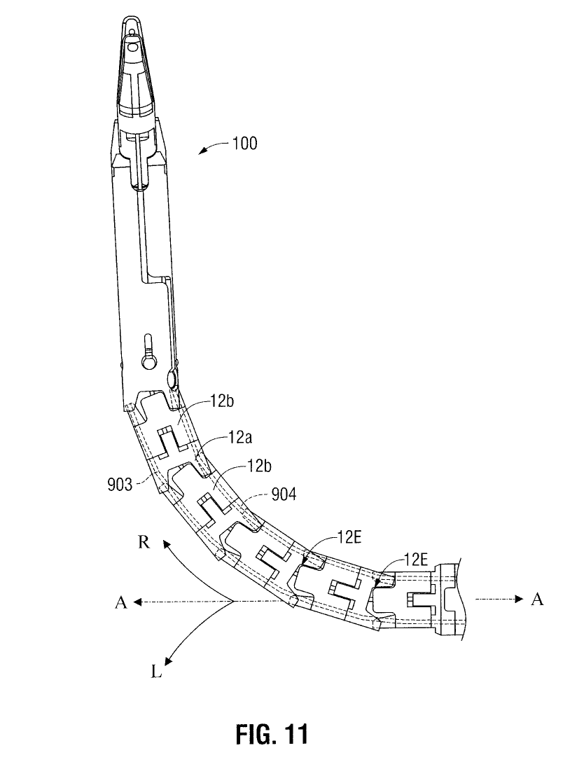

Referring now to FIG. 5 the articulation assembly 75 permits selective articulation of the end effector assembly 100 to facilitate the manipulation and grasping of tissue. More particularly, the two controls 80 and 90 include selectively rotatable wheels, 81 and 91, respectively, that sit atop the housing 20 (FIG. 1). Each wheel, e.g., wheel 81, is independently moveable relative to the other wheel, e.g., 91, and allows a user to selectively articulate the end effector assembly 100 in a given plane of articulation relative to the longitudinal axis A-A. For example, rotation of wheel 91 articulates the end effector assembly 100 along arrows R, L (or right-to-left articulation, see FIGS. 1 and 11) by inducing a differential tension and a corresponding motion in steering cables 903 and 904. Similarly, rotation of wheel 81 articulates the end effector assembly along arrows U, D (or up-and-down articulation, see FIGS. 1 and 13) by inducing a differential tension and a corresponding motion in steering cables 901 and 902.

Referring now to FIG. 6, the articulation assembly 75 includes an articulation block 250, which mounts longitudinally within the housing 20 (FIG. 1). Rotatable wheel 81 is operatively coupled to the articulation block 250 via an elongated hollow spindle 84. The spindle 84 is mechanically coupled at one end to the wheel 81 by a set-screw or a friction-fit, for example, such that rotation of the wheel 81 rotates the spindle 84. An opposite end of the spindle 84 interfaces similarly with a rotation beam 86 such that rotation of the spindle 84 effects rotation of the beam 86a relative to the articulation block 250. A beam plate 82 is attached to the articulation block 250 by bolts or other mechanical connections and prevents the beam 86 from sliding out of a receiving hole in the articulation block 250.

Beam 86, in turn, mounts to the articulation block 250 such that each end 86a and 86b couples to a respective slider 255a and 255b. Each slider 255a, 255b rides along a respective predefined rail 254a and 254b disposed in the articulation block 250. The sliders 255a and 255b each couple to an end of a respective steering cable 901 and 902 via a series of tensioning bolts 256a, 256b, sleeves 253a, 253b, washers 258a, 258b, elastic compression bushings or springs 259a, 259b and tensioning bolts 257a, 257b such that rotation of the wheel 81 in a given direction causes the respective sliders 255a and 255b to slide oppositely relative to one another within rails 254a and 254b to pull or stretch a respective steering cable 901, 902. For example, rotation of wheel 81 in a clockwise direction from the perspective of a user, i.e. in the direction of arrow 81d (DOWN “D”), causes the rotation beam 86 to rotate clockwise which, in turn, causes end 86a to rotate distally and end 86b to rotate proximally. Tensioning bolts 257a, 257b and bushings 259a, 259b are designed to maintain a general tension of the steering cables 901, 902 within the respective sliders 255a and 255b.

As a result thereof, as slider 255a moves distally and slider 255b moves proximally, steering cable 901 moves distally and steering cable 902 moves proximally, thus causing end effector assembly 100 to articulate DOWN “D”. The steering cable 902 may stretch as it moves longitudinally with respect to steering cable 901. When wheel 81 is rotated counter-clockwise, i.e. in the direction of arrow 81u, (UP “U”) the sliders 255a and 255b move in an opposite direction on rails 254a and 254b. The end effector assembly 100 is affected oppositely, i.e., the end effector assembly 100 is articulated in an UP “U” direction (See FIG. 13). Rotational movement of wheel 81 thus moves the end effector assembly 100 in an UP “U” and DOWN “D” plane relative to the longitudinal axis A-A (See FIG. 1). The cam-like connection between the sliders 255a and 255b and the beam 86 offers increased mechanical advantage when a user increases the articulation angle, i.e., the cam-like connection helps overcome the increasing resistance to articulation as the flexible portion 12b′ of shaft 12 is articulated in a given direction.

Rotatable wheel 91 of articulation control 90 is coupled to articulation block 250 in a similar manner. More particularly, wheel 91 operatively engages one end of a solid spindle 94 which, in turn, attaches at an opposite end thereof to rotation beam 96 disposed on an opposite end of the articulation block 250. Solid spindle 94 is dimensioned for insertion through hollow spindle 84 such that the solid spindle 94 is rotatable relative to the hollow spindle 84. Solid spindle 94 passes through the hollow spindle 84 and engages a locking nut 99. Locking nut 99 exhibits an outer profile that permits the locking nut 99 to seat within a locking recess 96′ engraved within rotation beam 96. Locking nut 99 is fixedly coupled to rotation beam 96 by welding or a similar process such that rotational motion of the solid spindle 94 is transferred to the rotation beam 96. Hollow spindle 84 exhibits an inner profile such that the solid spindle 94 has sufficient clearance to rotate therein without causing rotation of the hollow spindle 84.

Indexing wheels 87 and 97 are provided on either side of the articulation block 250. An internal bore extending through indexing wheel 87 is keyed to receive an end of hollow spindle 84 such that the indexing wheel 87 may rotate along with the hollow spindle 84. Likewise, an internal bore extending through indexing wheel 97 is keyed to receive an end of locking nut 99 such that indexing wheel 97 may rotate along with the locking nut 99, and thus, solid spindle 94. The exterior surfaces of indexing wheels 87, 97 include notches that interact with slides 265a, 265b, 265c and 265d to index the spindles 84, 94, and thus, wheels 81 and 91. The largest notch on the indexing wheels 87, 97 is designed to indicate a so-called “home” orientation for a respective rotatable wheel 81, 91. As the spindles 84 and 94 are rotated, the indexing wheels 87 and 97 act like miniature ratchet mechanisms to enhance fine discreet adjustment of each articulation wheel 81 and 91 relative to the longitudinal axis. Tensioning screws 263a, 263b, 263c and 263d and springs 262a, 262b, 262c and 262d are provided such that a force with which the slides 265a-265d engage the indexing wheels 87, 97 may be adjusted.

As mentioned above, spindle 94 extends through articulation block 250 to connect to rotation beam 96 (via locking nut 99). A beam plate 92 is utilized to secure the beam 96 to the articulation block 250. Much like beam 86, rotation beam 96 operably couples to a pair of sliders 255c and 255d, which are configured to ride in rails 254c and 254d defined in the articulation block 250. More particularly, each end 96a and 96b of beam 96 couples to a respective slider 255c and 255d. Thus, rotation of the beam 96 in a given direction causes the respective sliders 255c and 255d to slide oppositely relative to one another within rails 254c and 254d. The sliders 255c and 255d each couple to an end of a respective steering cable 903 and 904 via a series of tensioning bolts 256c, 256d, sleeves 253c, 253d, washers 258c, 258d, elastic compression bushings or springs 259c, 259d and tensioning bolts 257c, 257d. Thus, the movement of the sliders 255c and 255d tends to pull or contract respective steering cables 903, 904.

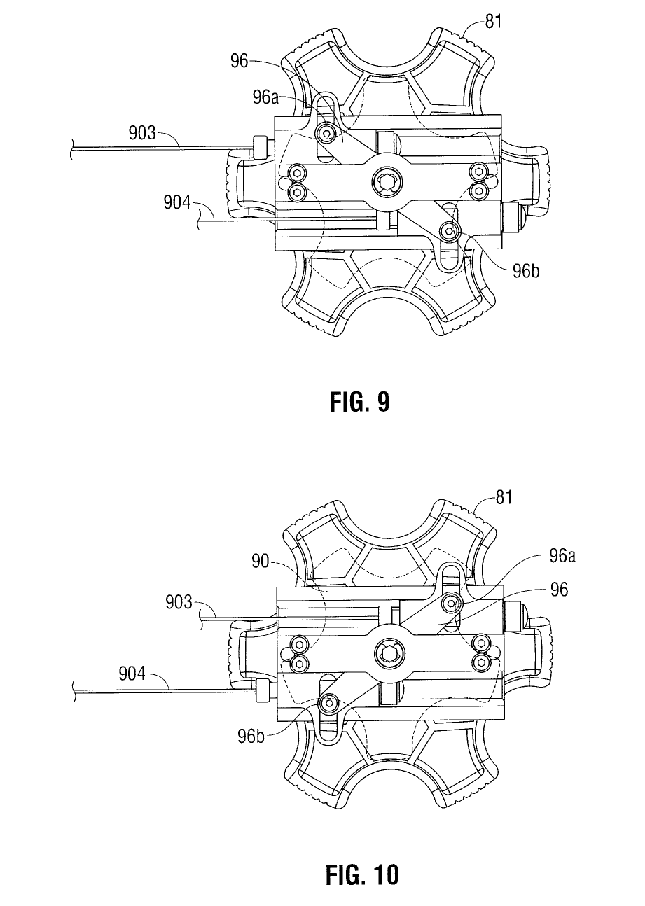

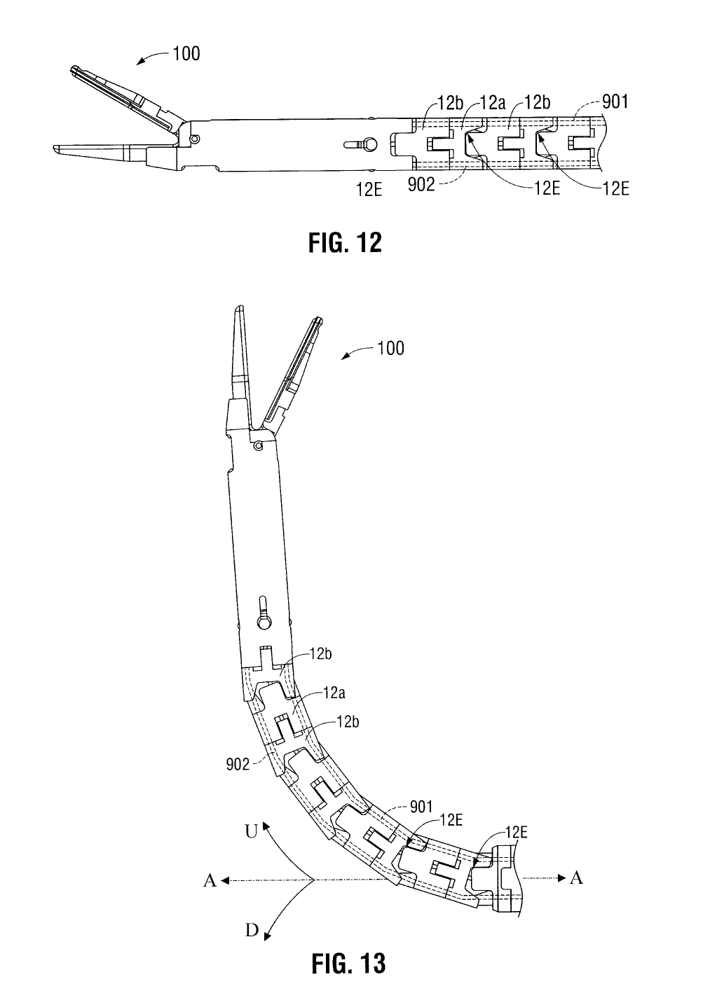

Rotation of wheel 91 in a clockwise direction from the perspective of a user, i.e., in the direction of arrow 91 (RIGHT “R”), causes the rotation beam 86 to rotate clockwise which, in turn, causes end 96a to rotate distally and end 96b to rotate proximally (See FIG. 9). As a result thereof, slider 255c moves distally and slider 255d moves proximally causing steering cable 903 to move distally and steering cable 904 to move proximally thus causing end effector assembly 100 to articulate to the RIGHT “R” (see FIG. 11). The steering cable 904 may stretch as it moves distally. When wheel 91 is rotated counter-clockwise, i.e. in the direction of arrow 91L, the sliders 255c and 255d move in an opposite direction on rails 254c and 254d (see FIG. 12) and end effector assembly 100 has an opposite effect, i.e., the end effector assembly 100 is articulated to the LEFT “L”. Rotational movement of wheel 91 moves the end effector assembly 100 in a RIGHT and LEFT plane relative to the longitudinal axis A-A.

As can be appreciated, the articulation assembly 75 enables a user to selectively articulate the distal end of the forceps 10 (i.e., the end effector assembly 100) as needed during surgery providing greater flexibility and enhanced maneuverability to the forceps 10 especially in tight surgical cavities. By virtue of the unique arrangement of the four (4) spring loaded steering cables 901-904, each articulation control 80 and 90 provides a positive drive, back and forth motion to the end effector assembly 100 that allows the end effector assembly 100 to remain in an articulated configuration under strain or stress as the forceps 10 is utilized, and/or prevent buckling of the elongated shaft 12 (FIG. 1) through a range of motion. Various mechanical elements may be utilized to enhance this purpose including the indexing wheels 87, 97 and the tensioning/locking mechanisms associated with slides 265a-265d. In addition, the flexible shaft 12 and end effector assembly 100 may also be manipulated to allow multi-directional articulation through the manipulation of both wheels 81 and 91 simultaneously or sequentially thereby providing more maneuverability to the forceps.

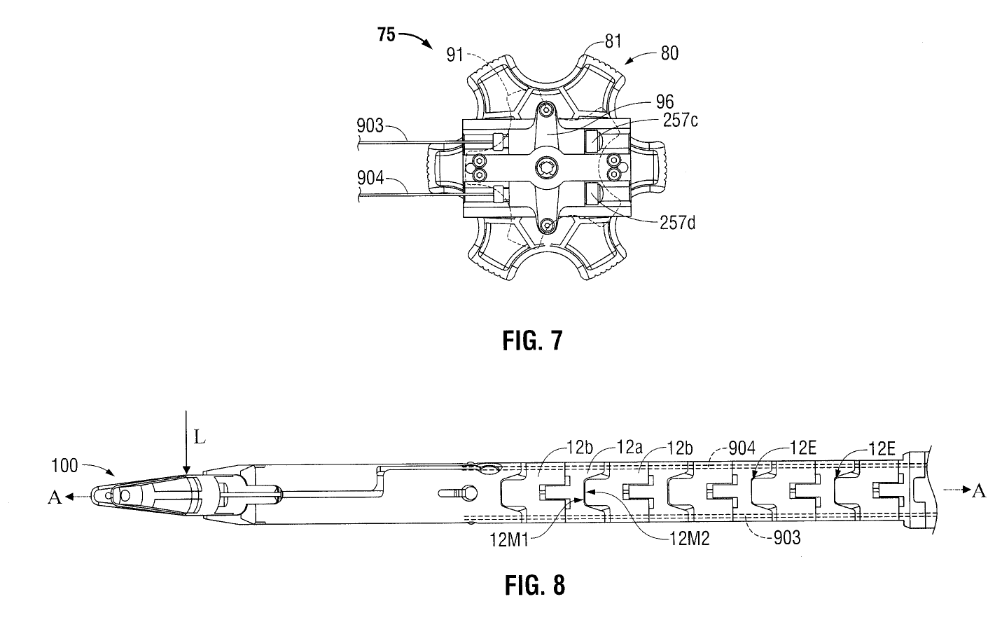

Referring now to FIGS. 7 and 8, the articulation assembly 75 may be moved to a “home” position to maintain the flexible portion 12b′ of shaft 12 in a non-articulated orientation aligned with the longitudinal axis A-A. When the articulation assembly 75 is moved to a “home” position for the RIGHT and LEFT plane, the rotation beam 96 is generally orthogonal to both of the steering cables 903 and 904. The steering cables 903 and 904, thus share a longitudinal position within the elongated shaft 12. A tension imparted to the steering cables 903, 904 by tensioning bolts 257c and 257d causes the steering cables 903, 904 to draw the end effector support 400 in a proximal direction and imparts a compressive force on the links 12a, 12b. Thus, links 12a and 12b maintain engagement about the substantially flat mating faces 12M1 and 12M2. The “home” position represents a state of minimum stored energy in the substantially elastic steering cables 903, 904 in which the collective stretching is least.

In use, if the end effector assembly 100 experiences a lateral load “L” the links 12a and 12b may resist a tendency to pivot relative to one another about edge 12E. The flat mating faces 12M1 and 12M2 provide a stable platform such that the tension in the steering cables 903 and 904 may maintain the links 12a and 12b in alignment with the longitudinal axis A-A. If however, the lateral load “L” is sufficient to overcome this tendency, the links 12a, 12b will pivot relative to one another and the end effector assembly 100 will articulate relative to the longitudinal axis A-A. The lateral load “L” will cause steering cable 904 to stretch and move relative to steering cable 903. The stretching of steering cable 904 increases the collective tension and stored energy of the steering cables 903, 904 as the end effector assembly 100 articulates. When the load “L” is removed, the links 12a and 12b will tend to return to the stable position where flat mating faces 12M1 and 12M2 are engaged and the collective stored energy in the steering cables 903 904 is at a minimum. In this regard, the links 12a and 12b may be regarded as “self-centering.”

Referring now to FIGS. 9-11, the articulation assembly 75 may be manipulated to articulate the end effector assembly 100 in the RIGHT and LEFT plane. As discussed above with reference to FIG. 5, the rotatable wheel 91 may be turned to move the steering cables 903 and 904. When the steering cable 903 is retracted proximally as depicted in FIG. 9, the end effector assembly 100 is articulated in the direction of arrow “R” as depicted in FIG. 11. Similarly, rotatable wheel 91 may be turned to retract steering cable 904 as depicted in FIG. 10 and thus articulate the end effector 100 in the direction of arrow “L”. Links 12a and 12b pivot relative to one another about rounded edges 12E defined by the links 12a. These edges 12E defined by links 12a, and about which the links 12a and 12b pivot to articulate the end effector assembly 100 in the RIGHT and LEFT plane, are oriented orthogonally to the RIGHT and LEFT plane.

Referring now to FIGS. 12 and 13, the edges 12E defined by the links 12b are oriented orthogonally to the UP and DOWN plane. Thus, the links 12a and 12b pivot relative to one another about the edges 12E defined by links 12b to articulate the end effector assembly 100 in the UP and DOWN plane. For example, steering cable 901 may be retracted by turning rotatable wheel 81 as described above with reference to FIG. 5. The end effector assembly 100 may thereby be articulated from a “home” position in the UP and DOWN plane as depicted in FIG. 12 to an articulated position in the direction of arrow “U” as depicted in FIG. 13. Similarly, the steering cable 902 may be retracted to induce articulation of the end effector assembly in the direction of arrow “D”.

The forceps 10 is suited for use by either a left or right-handed user and the articulation wheels 81 and 91 are particularly situated atop the housing 20 (FIG. 1) to facilitate usage thereof by either handed user. In another embodiment of a forceps (not shown), the entire shaft 12 (or portions thereof) may be flexible (or substantially flexible) along a length thereof to facilitate negotiation through a tortuous path. The number and size of the links 12a and 12b and end effector assembly 100 may be altered to meet a particular surgical purpose or to enhance effectiveness of the forceps 10 for a particular surgical solution.

In addition, it is also contemplated that one or more electrical motors may be utilized either automatically or manually to move the steering cables 901-904, advance the knife rod 504 or retract the drive rod 32. Although various cables, rods and shafts are employed for the various components herein, it is possible to substitute any one or all of these components with variations thereof depending upon a particular purpose.

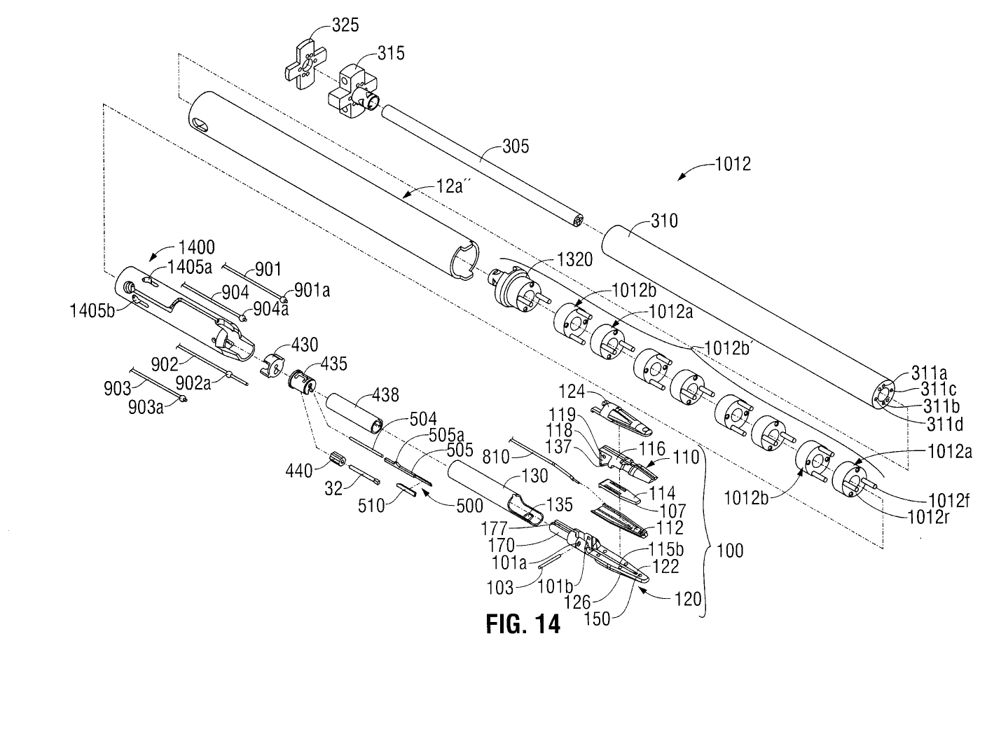

Referring now to FIG. 14, an alternate embodiment of an elongated shaft 1012 includes a flexible portion 1012b′. The flexible portion 1012b′ may be employed in place of flexible portion 12b′ of shaft 12 as described above with reference to FIG. 2. Flexible portion 1012b′ includes a plurality of links 1012a and 1012b. Each link 1012a engages a neighboring link 1012b such that the flexible portion 1012b′ may articulate the end effector assembly 100. Links 1012a are similar in construction to links 1012b in that each link 12a, 12b exhibits a substantially rigid base 1012r and a pair of relatively flexible tubes 1012f projecting from a distal face thereof. Links 1012a, however, are oriented with a ninety degree (90°) radial offset with respect to the neighboring link 1012b. Such an alternating orientation of the links 1012a, 1012b facilitates articulation of the end effector 100 in orthogonal planes. The four steering cables 901, 902, 903 and 904 are coupled to the articulation assembly 75 (FIG. 1) and extend through the flexible portion 1012b′ to induce articulation of the end effector assembly 100 as described in greater detail below.

Referring to FIG. 15, the substantially rigid base 1012r of link 1012a may be constructed of a metal such as stainless steel, or another material (e.g., ceramic or plastic) that is sufficiently rigid to retain its shape throughout normal surgical use of the instrument 10. The rigid base 1012r includes a central lumen 1019a extending longitudinally therethrough. The central lumen 1019a permits passage of various actuators, e.g., drive rod 32 and knife rod 504, and other components through the proximal portion 1012b′. Link 1012a also defines two pairs of opposed lumens 1017a and 1017b formed radially outward from the central lumen 1019a. Each of the lumens 1017a and 1017b on a link 1012a is radially spaced at a 90° from the neighboring lumen 1017a, 1017b such that each lumen 1017a aligns with a lumen 1017b of a neighboring link 1012b. The lumens 1017a and 1017b cooperate to define a longitudinal cavity to permit passage of the four steering cables 901, 902, 903 and 904 (FIG. 14) through the proximal portion 1012b′.

The relatively flexible tubes 1012f projecting from the base 1012r are received in opposed lumens 1017b. The tubes 1012f are constructed of an elastically deformable material such as spring steel or a shape-memory alloy. One particular alloy exhibiting a sufficient flexibility for the construction of the tubes 12f is nitinol, which is an alloy comprising titanium and nickel. The tubes 1012f may be press-fit or otherwise fixedly coupled to the base 1012r such that a passage 1019b defined through the tube 1012f is aligned with the lumen 1017b. The passage 1019b is sized sufficiently to permit the four steering cables 901, 902, 903 and 904 (FIG. 14) to slide therethrough. The flexible tubes 1012f are oriented to define a plane of articulation “P” orthogonal to a plane extending through the flexible tubes 1012f. As described below with reference to FIG. 16, the flexible tubes 1012f will bend more freely in a plane parallel with the plane of articulation.

Referring to FIG. 16, the tubes 12f projecting from the lumens 1017b of link 1012a are received within the lumens 1017a of a neighboring link 1012b. Thus, links 1012a are oriented with the ninety degree (90°) radial offset with respect to the neighboring link 1012b to define a pair of orthogonal bending directions. A first pair of tubes 1012f of link 1012a exhibit a tendency to bend more freely in the direction of arrows “U, D,” while a second pair of tubes 1012f of neighboring link 1012b tend to bend freely in the direction of arrows “R, L.”

Referring again to FIG. 14, a link support 1320 includes a pair of flexible tubes 1012f oriented similarly to a link 1012a to interface with a trailing link 1012b. A proximal end of the link support 1320 is fixedly mounted to outer casing 12a″, which extends over the proximal portion 1012a′ of the elongated shaft 12. An end effector support 1400 includes a two pairs of lumens (not shown) on a proximal end similar to the lumens 1017a and 1017b of a link 1012b to receive the flexible tubes 1012f of a leading link 1012a.

The four steering cables 901-904 may be substantially elastic and slideably extend through lumens pairs 1017a, and 1017b defined in the links 1012a and 1012b and the passages 1019b defined in the flexible tubes 1012f. A distal end of the each of the steering cables 901-904 is coupled to the end effector support 1400. More particularly, each steering cable 901-904 includes a ball-like mechanical interface as discussed above with reference to FIG. 2, namely, interfaces 901a-904a. Each interface 901a-904a is configured to securely mate within a corresponding recess defined in the end effector support 1400. Interface 904a engages recess 1405a, interface 903a engages recess 1405b, and interfaces 901a and 902a engage similar recess on the end effector support 1400.

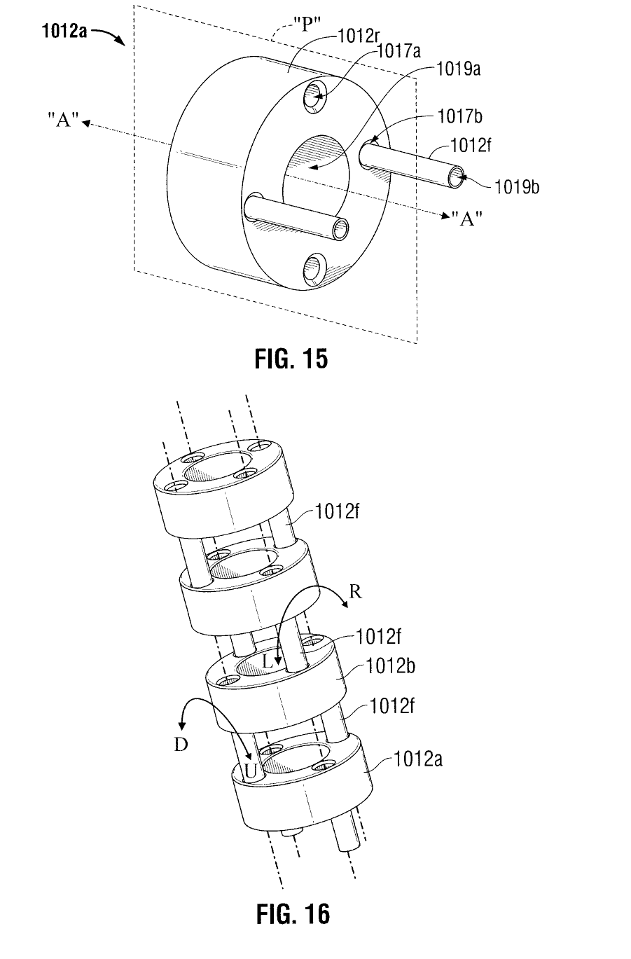

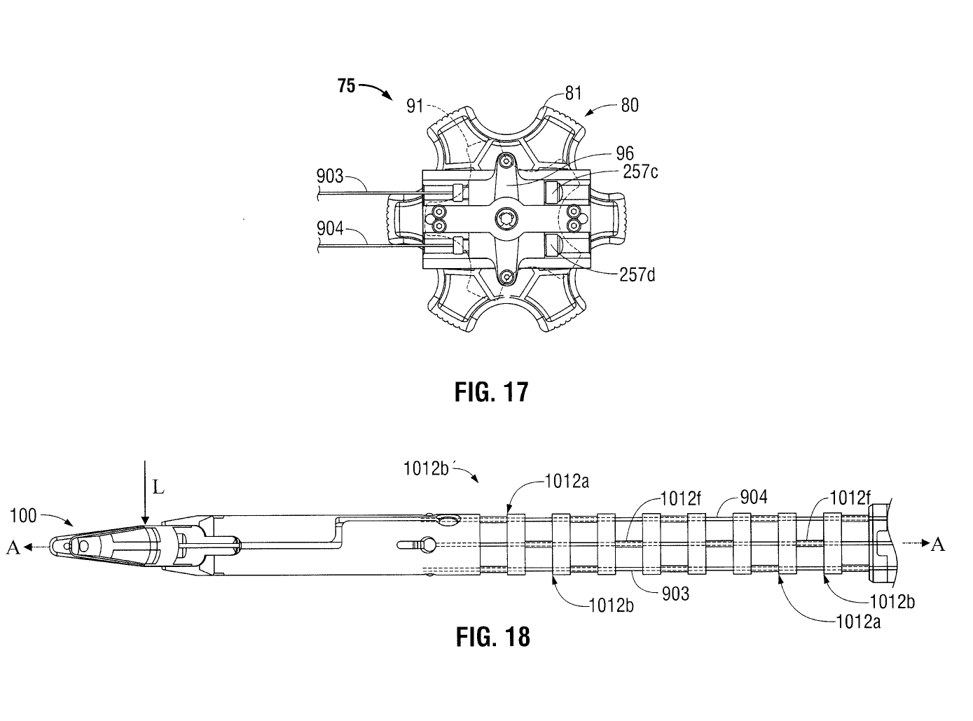

Referring now to FIGS. 17 and 18, the articulation assembly 75 may be moved to a “home” position to maintain the flexible portion 1012b′ in a non-articulated orientation aligned with the longitudinal axis A-A. When the articulation assembly 75 is moved to a “home” position for the RIGHT and LEFT plane, the rotation beam 96 is generally orthogonal to both of the steering cables 903 and 904. The steering cables 903 and 904, thus share a longitudinal position within the elongated shaft 12. A tension imparted to the steering cables 903, 904 by tensioning bolts 257c and 257d causes the steering cables 903, 904 to draw the end effector support 400 in a proximal direction and imparts a compressive force on the links 1012a, 1012b. This general tension reduces slack and play in the articulation assembly 75. The “home” position represents a state of minimum stored energy in the substantially elastic steering cables 903, 904 in which the collective stretching is least.

In use, if the end effector assembly 100 experiences a lateral load “L” the links 1012a and 1012b may resist a tendency to pivot relative to one another due to an inherent rigidity of the flexible tubes 1012f. The links 1012a and 1012b may thus maintain alignment with the longitudinal axis A-A. If however, the lateral load “L” is sufficient to overcome this tendency, the flexible tubes 1012f of links 1012b will bend and cause links 1012a, 1012b to pivot relative to one another. The end effector assembly 100 will thus articulate relative to the longitudinal axis A-A. The lateral load “L” will cause steering cable 904 to stretch and move relative to steering cable 903. The stretching of steering cable 904 increases the collective tension and stored energy of the steering cables 903, 904 as the end effector assembly 100 articulates. When the load “L” is removed, the links 1012a and 1012b will tend to return to the “home” position where the collective stored energy in the steering cables 903 904 is at a minimum. In this regard, the links 1012a and 1012b may be regarded as “self-centering.”

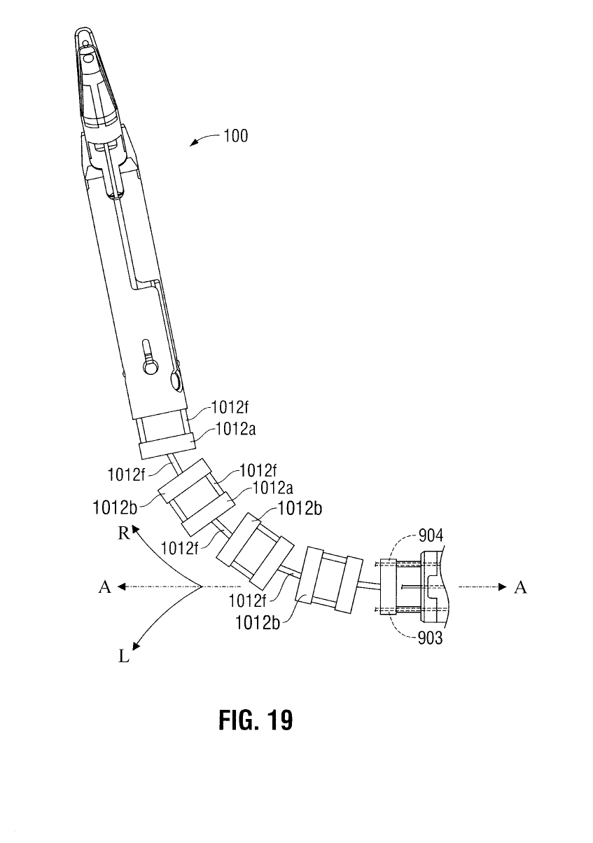

Referring now to FIG. 19, the articulation assembly 75 may be manipulated to articulate the end effector assembly 100 in the RIGHT and LEFT plane. As discussed above with reference to FIG. 5, the rotatable wheel 91 may be turned to move the steering cables 903 and 904. When the steering cable 904 is retracted proximally as depicted in FIG. 9, the end effector assembly 100 is articulated in the direction of arrow “R” as depicted in FIG. 19. The retraction of the steering cable 904 causes the flexible tubes 1012f of the links 1012b to bend in the direction of arrow “R.” Since the flexible tubes 1012f of the links 1012a lie in the RIGHT and LEFT plane, these flexible tubes may remain relatively straight. Similarly, rotatable wheel 91 may be turned to retract steering cable 903 as depicted in FIG. 10 and thus articulate the end effector 100 in the direction of arrow “L”.

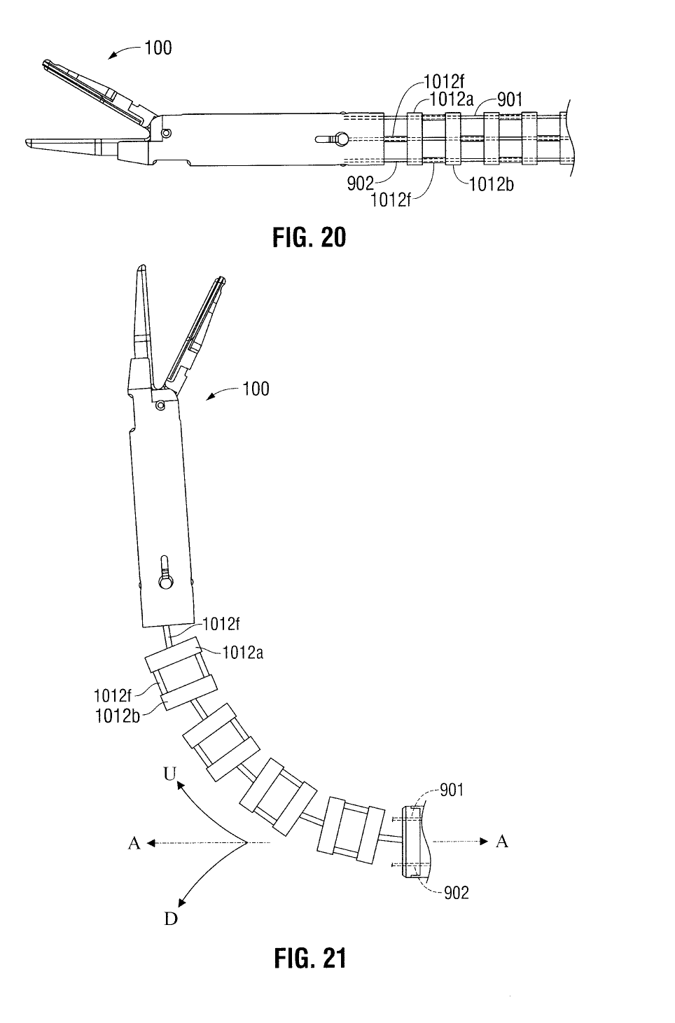

Referring now to FIGS. 20 and 21, the flexible tubes 1012f of links 1012a are oriented to bend to permit the links 1012a and 1012b pivot relative to one another to articulate the end effector assembly 100 in an UP and DOWN plane. For example, steering cable 901 may be retracted by turning rotatable wheel 81 as described above with reference to FIG. 5. The end effector assembly 100 may thereby be articulated from a “home” position in the UP and DOWN plane as depicted in FIG. 20 to an articulated position in the direction of arrow “U” as depicted in FIG. 21. Similarly, the steering cable 902 may be retracted to induce articulation of the end effector assembly in the direction of arrow “D”.

Referring now to FIG. 22, links 1012c and 1012d may be assembled to form an alternate embodiment of a flexible portion of an articulating shaft. Links 1012c and 1012d are similar in construction, but are oriented with a ninety degree (90°) radial offset with respect to one another. Similarly to the links 1012a and 1012b described above with reference to FIGS. 15 and 16, links 1012c and 1012d each exhibit a substantially rigid base 1012r and a pair of relatively flexible tubes 1012f projecting from a proximal face thereof. Lumens 1017a and 1017b permit passage of steering cables 901, 902, 903, and 904 to induce bending of the tubes 1012f, and thus articulation of the links 1012c and 1012d in the orthogonal bending directions indicated by the arrows “R, L” and “U, D.”

Links 1012c and 1012d include a set of distal ribs 1012R1 projecting from a distal face of the substantially rigid base 1012r, and a set of proximal ribs 1012R2 projecting from a proximal face of the base 1012R. The distal ribs 1012R1 each include a sliding face 1012S1 that is parallel to the bending direction defined by the tubes 1012f of the link 1012c or 1012d. The proximal ribs 1012R2 each include a sliding face 1012S2 that is perpendicular to the bending direction. When a link 1012c is assembled adjacent a neighboring link 1012d with a ninety degree (90°) radial offset, the sliding faces 1012S1 and 1012S2 slide past one another as the tubes 1012f bend. The distal and proximal ribs 1012R1, 1012R2 engage each other such that the links 1012c and 1012d may resist torsional loads. If a torsional load is applied in the direction of arrows “T,” the ribs 1012R1, 1012R2 will prevent radial displacement of the links 1012c and 1012d in the same direction. Thus the ribs 1012R1, 1012R2 may assist in positioning an end effector assembly 100 (FIG. 1) by ensuring that the relative motion between the links 1012c and 1012d remains along the bending directions anticipated by a surgeon and controllable using articulation assembly 75 (FIG. 1).

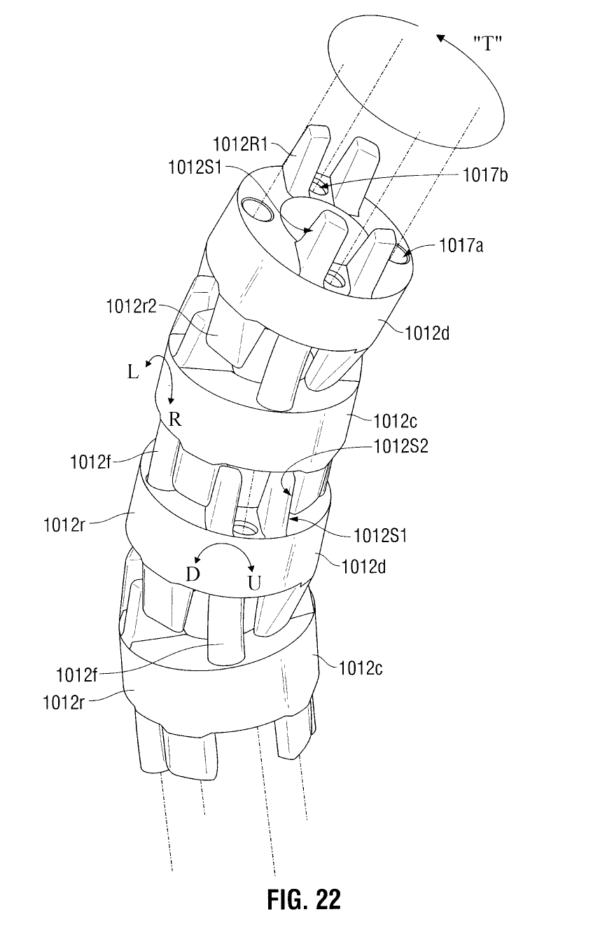

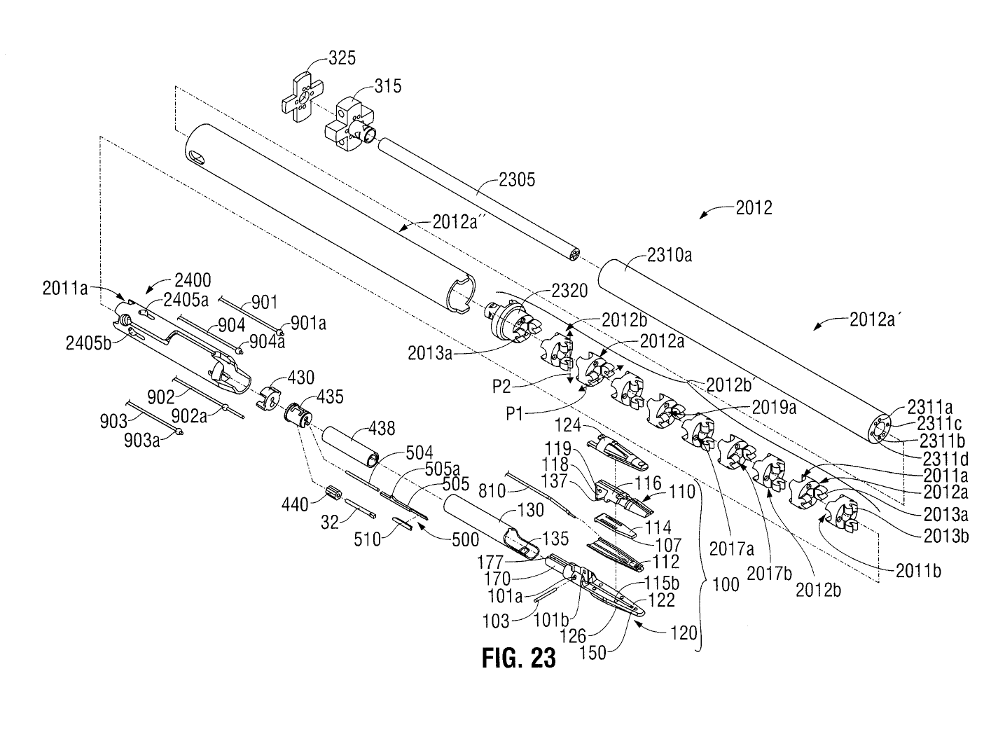

Referring now to FIG. 23, another alternate embodiment of an elongated shaft 2012 includes a proximal portion 2012a′ and a distal articulating portion 2012b′. The proximal and distal portions 2012a′ and 2012b′ may be employed in place of proximal and distal portions 12a′ and 12b′ of shaft 12 as described above with reference to FIG. 2. Articulating distal portion 2012b′ includes a plurality of links 2012a and 2012b. Each link 2012a engages a neighboring link 2012b such that the distal portion 2012b′ may articulate the end effector assembly 100. Links 2012a are similar in construction to links 2012b in that each link 2012a, 2012b exhibits a pair of distal knuckles 2013a, 2013b and pair of opposing proximal devises 2011a, 2011b formed therewith. Links 2012a, however, are oriented with a ninety degree (90°) radial offset with respect to the neighboring link 2012b. Such an alternating orientation of the links 2012a, 2012b facilitates articulation of the end effector 100 in orthogonal planes. The distal knuckles 2013a of links 2012a define a horizontal pivot axis P1. Thus a distal knuckle 2013a operatively engages a corresponding clevis 2011b of a neighboring link 2012b to facilitate articulation of the end effector 100 in the direction of arrows “U, D” (FIG. 1). Similarly, the distal knuckles 2013b of links 2012b define a vertical pivot axis P2 such that a distal knuckle 2013b operatively engages a corresponding clevis 2011a of a neighboring link 12a to facilitate articulation of the end effector 100 in the direction of arrows “R, L.”

Each link 2012a and 2012b includes a central lumen 2019a extending longitudinally therethrough. The central lumen 2019a permits passage of various actuators, e.g., drive rod 32 and knife rod 504, and other components through the articulating distal portion 2012b′. Links 2012a, 2012b also define two pairs of opposed lumens 2017a and 2017b formed radially outward from the central lumen 2019a. Each of the lumens 2017a and 2017b on a link 2012a is radially spaced at a 90° from the neighboring lumen 2017a, 2017b such that each lumen 2017a aligns with a lumen 2017b of a neighboring link 2012b. The lumens 2017a and 2017b cooperate to define a longitudinal cavity to permit passage of four steering cables 901, 902, 903 and 904 through the articulating portion 2012b′. A differential tension may be imparted to the four steering cables 901-904 to adjust the orientation of the articulating distal portion 2012b′ of shaft 2012 as described below with reference to FIGS. 31, 33 and 35.

A link support 2320 includes a pair of distal knuckles 2013a oriented similarly to a link 2012a to interface with a trailing link 2012b. A proximal end of the link support 2320 is fixedly mounted to an outer casing 2012a″, which extends over the proximal portion 2012a′ of the elongated shaft 2012. The outer casing 2012a″ is generally flexible to permit the proximal portion 2012a′ to flex and bend freely. An end effector support 2400 includes a pair of devises 2011a on a proximal end oriented similarly to a link 2012a to receive the distal knuckles 2013b of a leading link 2012b.

The four steering cables 901-904 may be substantially elastic and slideably extend through lumens pairs 2017a, and 2017b defined in the links 2012a and 2012b. A distal end of the each of the steering cables 901-904 is coupled to end effector support 2400. More particularly, each steering cable 901-904 includes a ball-like mechanical interface at the distal end, namely, interfaces 901a-904a. Each interface 901a-904a is configured to securely mate within a corresponding recess defined in the end effector support 2400. Interface 904a engages recess 2405a, interface 903a engages recess 2405b, and interfaces 901a and 902a engage similar recess on the end effector support 2400.

Proximal ends of the steering cables 901-904 are operatively coupled to the articulation controls 80, 90 as described below with reference to FIGS. 5 and 6. The steering cables 901-904 extend through the shaft 2012 through a series of passageways defined therein. More particularly, cross-shaped cable guide adapter 315 and guide adapter liner or washer 325 include bores defined therethrough to initially orient the cables 901-904 at 90° degree angles relative to one another for passage into an outer tube 2310A. The adapter 315 may also facilitate attachment of the shaft 2012 to the housing 20. The tube 2310A includes passageways 2311a-2311d defined therein to orient the cables 901-904, respectively, for reception into the lumens 2017a, 2017b of links 2012a and 2012b for ultimate connection to the end effector support 2400 as described above. The tube 2310A exhibits a composite construction as described below with reference to FIG. 24. The composite construction of tube 2310A facilitates maintenance of a non-aligned shape and orientation of the proximal portion 2012a′ of the shaft 2012 as tensile forces in the cables 901-904 are transferred to the tube 2310A.

A central guide tube 2305 is provided to orient the drive rod 32 and the knife rod 504 through the shaft 2012 for ultimate connection to jaw member 110 and a knife assembly 500. The central guide tube 305 also guides an electrical lead 810 for providing electrosurgical energy to the jaw member 110. The central guide tube 2305 is dimensioned for reception within outer tube 2310A, and may extend distally therefrom into the central lumens 2019a defined in the links 2012a and 2012b. One or more steering cables, e.g., 902, includes a distal portion 902b that electrically connects to the end effector support 2400 which, in turn, connects to jaw member 120. A return path (i.e., ground path) may thus be established through tissue captured between jaw members 110 and 120 for electrosurgical energy provided through jaw member 110.

The central extrusion or guide tube 2305 is constructed from a highly flexible and lubricious material and performs several important functions: tube 2305 guides the drive rod 32, the knife rod 504 and the electrical lead 810 from the guide adapter 315, through the shaft 2012 to the end effector support 2400 and knife assembly 500; the tube 2305 provides electrical insulation between component parts; the tube 2305 keeps the lead 810 and rods 32 and 504 separated during relative movement thereof; the tube 2305 minimizes friction and clamping force loss; and tube 2305 keeps the lead 810 and rods 32 and 504 close to the central longitudinal axis to minimize stretching during articulation. The tube 2305 (and internal lumens) may be made from or include materials like polytetrafluoroethene (PTFE), graphite or other lubricating agents to minimize friction and other common losses associated with relative movement of component parts. Alternatively, a coaxial structure (not shown) may be utilized to guide the drive rod 32 and knife rod 504.

Many of the components of shaft 2012 may be identical in construction and operation as corresponding components discussed above. For example, many of the components disposed distally of the end effector support 2400 correspond to components of shaft 12 described above with reference to FIG. 2 and shaft 1012 described above with reference to FIG. 14.

Referring now to FIG. 24, the tube 2310A includes two concentric extrusions. An outer tubular layer 2312 is relatively thick and flexible, while an inner tubular core layer 2314 is relatively thin and rigid. The outer layer 2312 defines an outer diameter OD₁, and may be constructed of a soft thermoplastic elastomer such as PEBAX® 7033, available from the Arkema, Group Technical Polymers Unit in Colombes, France. The inner core layer 2314 defines an inner diameter ID₁, and may be constructed of a thin tube of a metal such as superelastic nitinol. An intermediate, or medial diameter MD₁ is defined at the boundary of the inner core layer 2314 and the outer layer 2312. In one example, where ID₁=0.128 inches, MD₁=0.142 inches and OD₁=0.300 inches, the outer layer 2312 defines a first wall thickness of about 0.079 inches. The inner layer defines a second wall thickness of about 0.007 inches, or about nine percent of the first wall thickness. In some embodiments, the inner core layer 2314 defines a wall thickness that is 5% to 15% of the wall thickness of outer layer 2312. This arrangement provides a flexible shaft with appropriate axial and flexural rigidities for use in a surgical instrument.

The axial rigidity EA₁ of the tube 2310A may be expressed as EA₁=E′A′+E″A″ where E′ is the modulus of elasticity for the outer layer 2312, A′ is the cross-sectional area of the outer layer 2312, E″ is the modulus of elasticity of the outer layer and A″ is the cross-sectional area of the outer layer 2312. Assuming that the cross-sectional area in the outer layer 2312 occupied by the lumens 901-904 is negligible, the axial rigidity EA₁ of the tube 2310A may be expressed as:

EA₁=E′·(π·(OD₁/2)²−π·(MD₁/2)²)+E″·(π·(MD₁/2)²−π·(ID₁/2)²).

Substituting the values listed above for various diameters, a value of E′=52,600 psi for the modulus of elasticity for PEBAX® 7033, an approximate value of E″=6×10⁶ psi for the modulus of elasticity for nitinol and estimating π=3.14, the axial rigidity EA₁ of the tube 2310A may be determined.

EA₁=52,600 psi·(π·(0.300 in/2)²−π(0.142 in/2)²)+6×10⁶ psi·(π·(0.142 in/2)²−π·(0.128 in/2)²), or

EA₁=20,688 lbs.

This axial rigidity EA₁ is relatively high such that the tube 2310A may resist deformation under axial loads. The flexural rigidity EI₁ of tube 2310A, however, remains relatively low. The flexural rigidity EI₁ may be expressed as EI₁=E′·I′+E″·I″ where E′ and E″ are the modulus of elasticity values expressed above, I′ is the cross-sectional moment of inertia of the outer layer 2312 and I″ is the cross-sectional moment of inertia of the inner core layer 2314. The formula for the cross-sectional moment of inertia for an annulus of I₀=(π/64)·(D_{O}⁴−D_{I}⁴), where D_{O} is the outer diameter and D_{I} is the inner diameter, may be used to calculate values for I′ and I″. Thus, the flexural rigidity EI₁ of the tube 310A may be expressed as

EI₁=E′·(π/64)·(OD₁⁴−MD₁⁴)+E″·(π/64)·(MD₁⁴−ID₁⁴), or

EI₁=52,600 psi·(π/64)·((0.300 in)⁴−(0.142 in)⁴)+6×10⁶ psi·(π/64)·((0.142)⁴−(0.128 in)⁴),

or

EI₁=19.9 lb·in²+40.7 lb·in², or

EI₁=60.6 lb·in².

This flexural rigidity is relatively low such that the tube 2310A may be conformable to facilitate positioning of the end effector 100 (FIG. 23) at a surgical site. The values computed for the axial and flexural rigidities are respectively high and low as compared to the corresponding values for a suitable tube with similar envelope dimensions, but having a uniform construction.

Referring now to FIG. 25, tube 2310B exhibits a uniform construction having an outer diameter OD₂=0.300 in and an inner diameter ID₂=0.128 in, similar to the tube 2310A described above with reference to FIG. 24. The tube 2310B is constructed of Nylon 12 having a modulus of elasticity of about E=186,000 psi. The axial rigidity EA₂ of the tube 310B may be expressed as

EA₂=E·(π(OD₂/2)²−π·(ID₂/2)²), or

EA₂=186,000 psi·(π·(0.300 in/2)²−π·(0.128 in/2)²), or

EA₂=10,748 lb.

This axial rigidity EA₂ of the tube 2310B is only about half of the axial rigidity EA₁ of the tube 2310A. The flexural rigidity EI₂ of the tube 2310B may be expressed as

EI₂=E·(π/64)·(OD₂⁴ID₂⁴), or

EI₂=186,000 psi·(π/64)·((0.300 in)⁴−(0.0128 in)⁴), or

EI₂=71.5 lb·in².

The flexural rigidity EI₂ of the tube 2310B is significantly higher than the flexural rigidity EI₁ of the tube 2310A. Thus, the composite structure of tube 2310A offers improvements over the uniform construction of tube 2310B in both the axial and flexural rigidities.

More traditional methods of increasing the axial rigidity EA₂ of a tube 2310B include increasing the modulus of elasticity E, or increasing the outer diameter OD₂. Selecting a material having an increased modulus of elasticity E, however, also increases the flexural rigidity EI₂ of the tube 310B by the same degree. Consequently, the tube 2310B is less conformable to navigate curved or tortuous paths. Similarly, increasing the outer diameter OD₂ yields undesirable consequences. Increasing the outer diameter OD₂ by 10% yields a 27% increase in the axial rigidity EA₂, but also yields a 50% increase in the flexural rigidity EI₂. Again, increasing the outer diameter OD₂ yields a tube 2310B that is less conformable to navigate tortuous paths, and also a tube 2310B that is simply larger and less suitable for endoscopic surgical procedures.

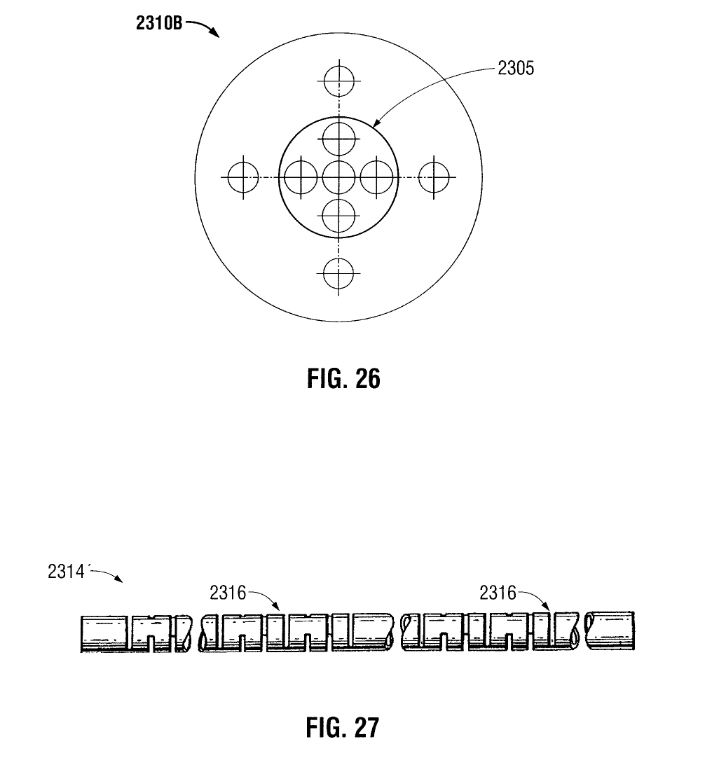

Referring now to FIG. 26, the tube 2310B, may be positioned over central guide tube 2305 to provide additional axial rigidity. A relatively rigid material may be selected for central guide tube 2305 that exhibits a higher modulus of elasticity than the modulus of elasticity E of nylon 12. Where the central guide tube 2305 is constructed of a relatively rigid material, however, the central guide tube 2305 should not extend into the central lumens 2019a defined in the links 2012a and 2012b so as not to inhibit the articulation of the distal shaft portion 2012b′.

Other embodiments of a tube member for the construction of a flexible portion of an endoscopic shaft are envisioned. For example, a tube with three or more layers may be designed to suit a particular purpose. Each layer may have a different modulus of elasticity than the neighboring layers to appropriately balance the axial and flexural rigidities. The various layers may provide additional benefits or perform additional functions. For example, one or more of the layers may be configured to conduct electricity or reduce friction as shaft bends.

In another embodiment, an inner layer may be constructed from a stainless steel tube rather than the superelastic nitinol discussed above with reference to FIG. 24. The stainless steel tube may include laser cuts therein to minimize flexural rigidity. For example, the tube 2314′ depicted in FIG. 26 includes a series of laterally-oriented, laser cut notches 2316 formed therein in a helical pattern. This arrangement provides a high axial rigidity and a low flexural rigidity since the notches 2316 permit lateral bending. In yet other embodiments, an anisotropic tube may be provided wherein the modulus of elasticity generally or gradually decreases as a function of the radius.

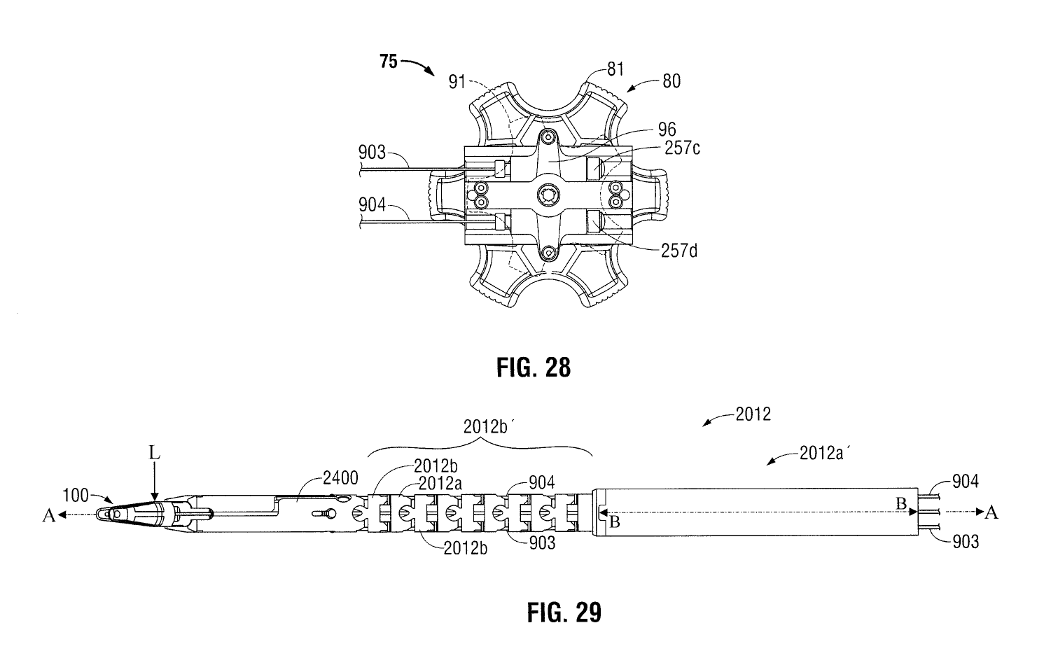

Referring now to FIGS. 28 and 29, the articulation assembly 75 may be moved to a “home” position to maintain the articulating portion 2012b′ of shaft 2012 in a non-articulated orientation aligned with the proximal shaft axis B-B. The flexible proximal portion 2012a′ of the elongated shaft 2012 is aligned with the longitudinal axis A-A. When the articulation assembly 75 is moved to a “home” position for the RIGHT and LEFT plane, the rotation beam 96 is generally orthogonal to both of the steering cables 903 and 904. The steering cables 903 and 904, thus share a longitudinal position within the elongated shaft 12. A tension imparted to the steering cables 903, 904 by tensioning bolts 257c and 257d causes the steering cables 903, 904 to draw the end effector support 2400 in a proximal direction and imparts a compressive force on the links 2012a, 2012b. This general tension reduces slack and play in the articulation assembly 75. The “home” position represents a state of minimum stored energy in the substantially elastic steering cables 903, 904 in which the collective stretching is least.

In use, if the end effector assembly 100 experiences a lateral load “L” the links 2012a and 12b may resist a tendency to pivot relative to one another due to the general tension in the steering cables 903, 904. The links 2012a and 2012b may thus maintain alignment with the proximal shaft axis B-B. If however, the lateral load “L” is sufficient to overcome this tendency, the links 2012a will pivot relative to neighboring links 2012b to cause the end effector assembly 100 to articulate relative to the proximal shaft axis B-B. The lateral load “L” will cause steering cable 904 to stretch and move relative to steering cable 903. The stretching of steering cable 904 increases the collective tension and stored energy of the steering cables 903, 904 as the end effector assembly 100 articulates. When the load “L” is removed, the links 2012a and 2012b will tend to return to the “home” position where the collective stored energy in the steering cables 903 904 is at a minimum. In this regard, the links 2012a and 2012b may be regarded as “self-centering.”

Referring now to FIG. 30, the flexible proximal portion 2012a′ of the elongated shaft 2012 may be shaped to assume a curve to the left from the perspective of a user. Establishing such a curve is facilitated by the relatively low flexural rigidity of the tube 2310A that supports the proximal portion 2012a. When such a curve is established, the proximal shaft axis B-B diverges from the longitudinal axis A-A. The relatively high axial rigidity of the tube 2310A facilitates maintenance of the curve under the influence of the general tension in the steering cables, e.g., 903 and 904.

In contrast to the proximal shaft portion 2012a′ supported by a tube 2310A having a composite construction, proximal shaft portion 2012a′2 depicted in FIG. 31 provides a tube having a uniform construction with an insufficient axial rigidity. When the links 2012a and 2012b are pivoted relative to one another to curve the distal portion 2012b′ to the right, the proximal portion 2012a′2 tends to return to a straightened configuration. This straightening may frustrate the intent of a surgeon intending to maintain a curve in the proximal portion 2012a′2.

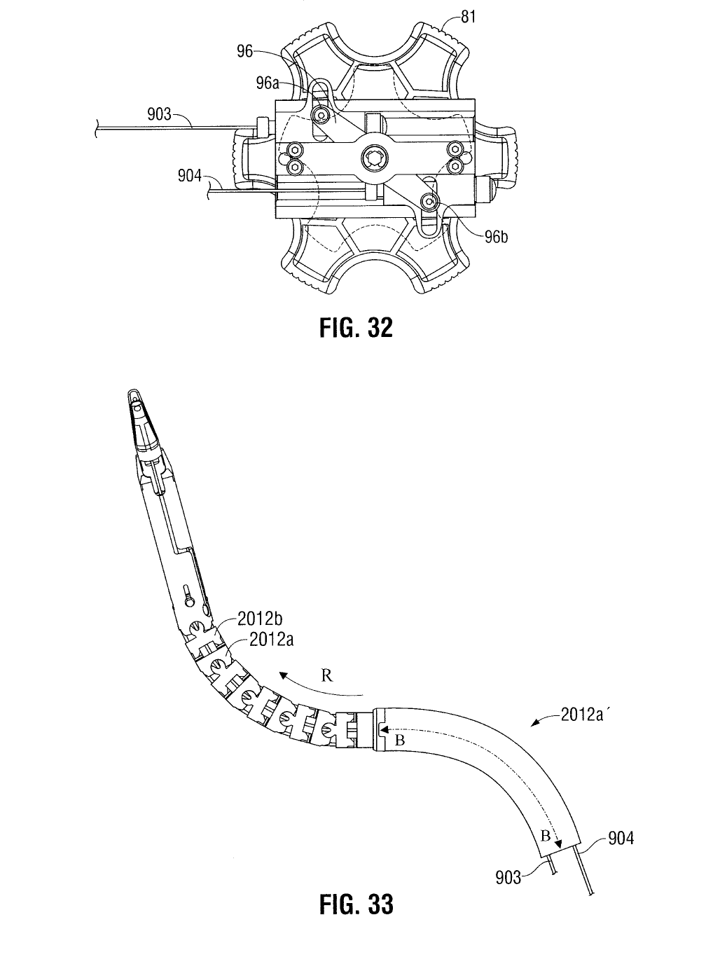

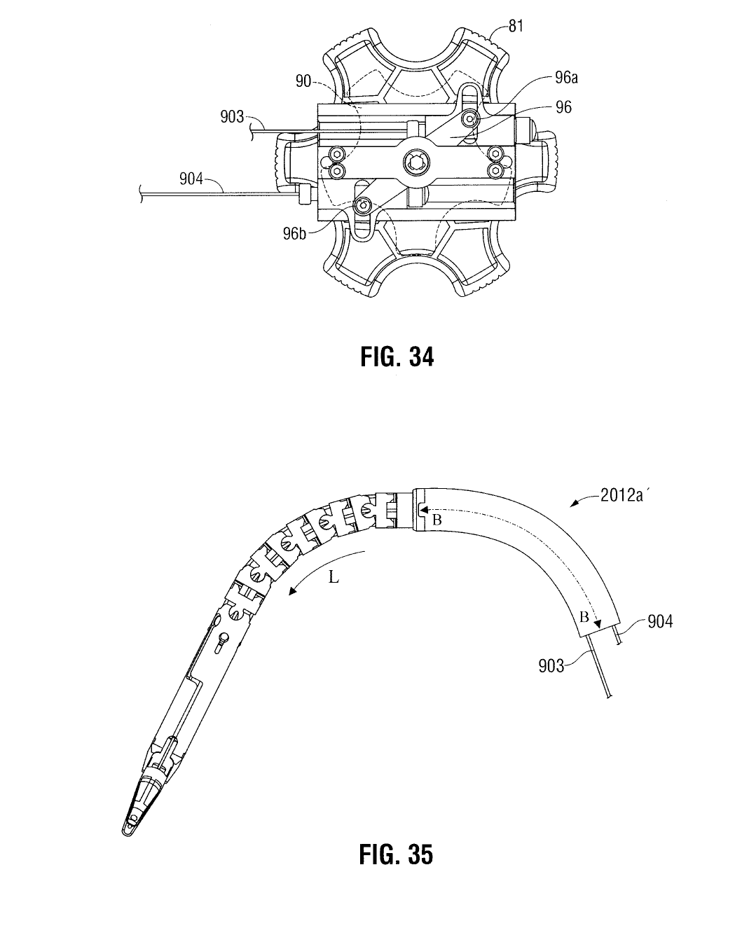

Referring now to FIGS. 32 and 33, the steering cables 903, 904 permits articulation assembly 75 to be manipulated to articulate the end effector assembly 100 in the RIGHT and LEFT plane regardless of the curvature of the proximal portion 2012a′. As discussed above with reference to FIG. 5, the rotatable wheel 91 may be turned to move the steering cables 903 and 904. When the steering cable 904 is retracted proximally as depicted in FIG. 32, the end effector assembly 100 is articulated in the direction of arrow “R” with respect to the proximal shaft axis B-B as depicted in FIG. 33. The retraction of the steering cable 904 causes the links 2012a to pivot relative to neighboring links 2012b in the direction of arrow “R.” Similarly, rotatable wheel 91 may be turned to retract steering cable 903 as depicted in FIG. 34 and thus articulate the end effector 100 in the direction of arrow “L” as depicted in FIG. 35. The curvature in the proximal portion 2012a′ is maintained due to the axial rigidity of the tube 2310A (FIG. 24).

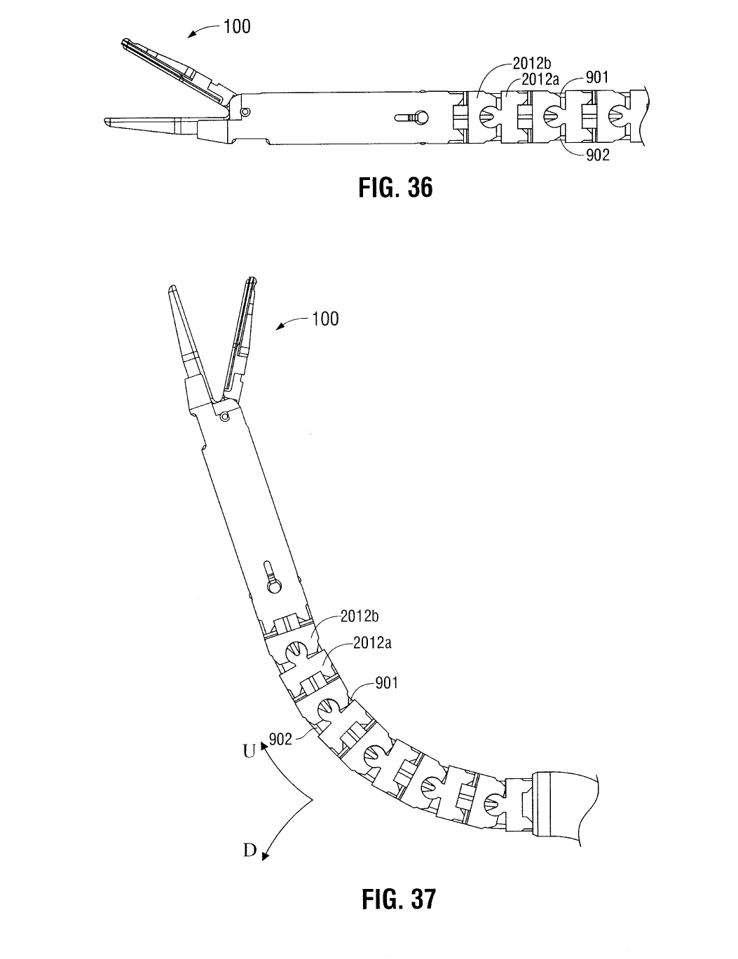

Referring now to FIGS. 36 and 37, the radial offset between links 2012a and 2012b permit the end effector assembly 100 to articulate in an UP and DOWN plane as well. For example, steering cable 901 may be retracted by turning rotatable wheel 81 as described above with reference to FIG. 5. The end effector assembly 100 may thereby be articulated from a “home” position in the UP and DOWN plane as depicted in FIG. 36 to an articulated position in the direction of arrow “U” as depicted in FIG. 37. Similarly, the steering cable 902 may be retracted to induce articulation of the end effector assembly in the direction of arrow “D”.

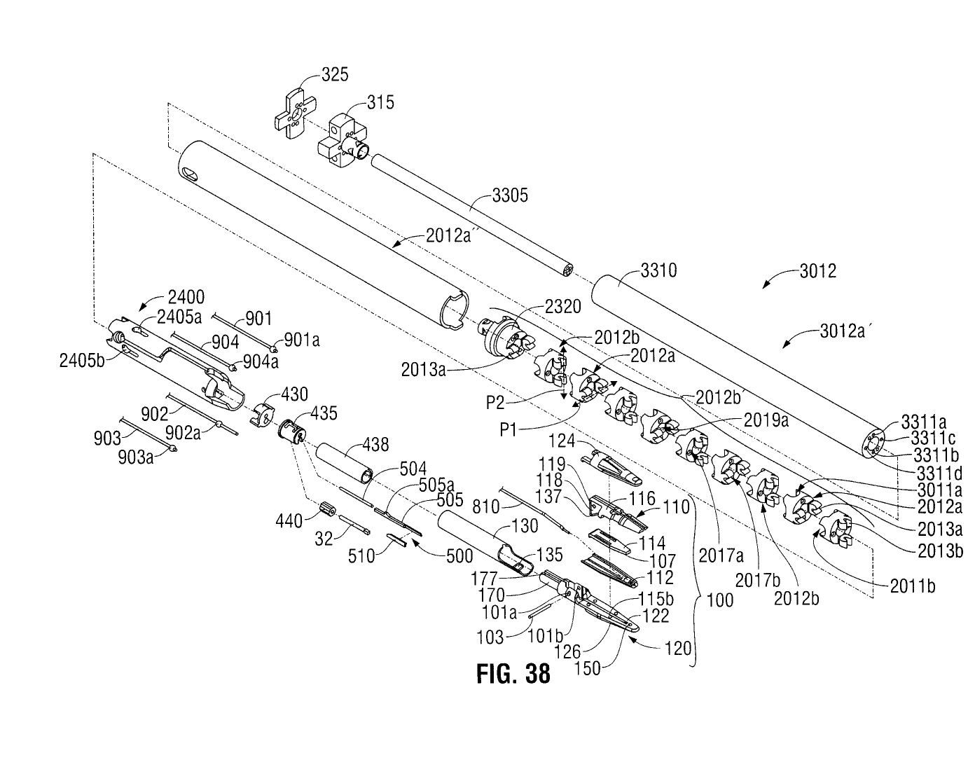

Referring now to FIG. 38, another alternate embodiment of an elongated shaft 3012 includes a proximal portion 3012a′. The proximal portions 3012a′ may be employed in place of proximal portions 2012a′ as described above with reference to FIG. 23. The elongated shaft 3012 includes distal articulating portion 2012b′ as described above with reference to FIG. 23, although other distal articulating portions 12b′ (FIG. 2) or 1012b′ (FIG. 14) may be employed.

Proximal ends of the steering cables 901-904 are again operatively coupled to the articulation controls 80, 90 as described below with reference to FIGS. 5 and 6. The steering cables 901-904 extend through the shaft 3012 through a series of passageways defined therein. More particularly, cross-shaped cable guide adapter 315 and guide adapter liner or washer 325 include bores defined therethrough to initially orient the cables 901-904 at 90° degree angles relative to one another for passage into an outer tube 3310. The adapter 315 may also facilitate attachment of the shaft 3012 to the housing 20. The tube 3310 includes passageways 3311a-3311d defined therein to orient the cables 901-904, respectively, for reception into the lumens 2017a, 2017b of links 2012a and 2012b for ultimate connection to the end effector support 2400 as described above. A central guide tube 3305 is utilized to orient the drive rod 32 and the knife rod 504 through the shaft 3012 for ultimate connection to jaw member 110 and a knife assembly 500 in a manner similar to guide tube 2305 described above with reference to FIG. 23.

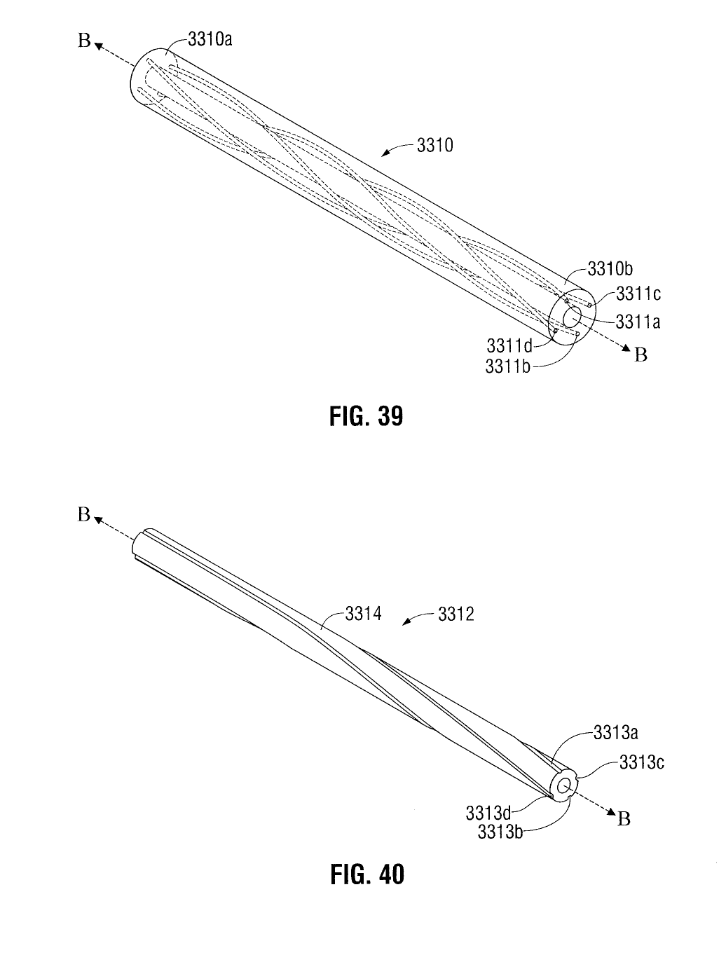

Referring now to FIG. 39, the passageways 3311a-3311d of tube 3310 are helically arranged around the proximal shaft axis B-B. Each passageway 3311a-3311d traverses a full radial arc, i.e. 360°, between a proximal end 3310a and a distal end 3310b of the tube 3310. This arrangement permits each of the four steering cables 901-904 to exhibit the same radial orientation immediately distally of the tube 3310 as immediately proximal to the tube 3310.

In alternate embodiments, such as tube 3312 depicted in FIG. 40, passageways 3313a-3313d may traverse a radial arc of 180° such that each of the four steering cables 901-904 exhibits an opposite radial orientation immediately distally of the tube 3312 as immediately proximal to the tube 3312. The radial arc is an increment of 180° such that an approximately equal longitudinal length of each passageway 3313a-3313d is disposed on each of two opposed lateral sides of the proximal shaft axis B-B. The passageways 3313a-3313d define grooves in an exterior surface 3314 of the tube 3312. A cover tube (not shown) may be provided to encircle the exterior surface 3314 and maintain the steering cables 901-904 in a corresponding passageway 3313a-3313d.

Referring now to FIGS. 41 and 42, the articulation assembly 75 may be moved to a “home” position to maintain the articulating portion 2012b′ of shaft 3012 in a non-articulated orientation aligned with the proximal shaft axis B-B. The flexible proximal portion 2012a′ of the elongated shaft 3012 is aligned with the longitudinal axis A-A. When the articulation assembly 75 is moved to a “home” position for the RIGHT and LEFT plane, the rotation beam 96 is generally orthogonal to both of the steering cables 903 and 904. The steering cables 903 and 904, thus share a longitudinal position within the elongated shaft 3012.

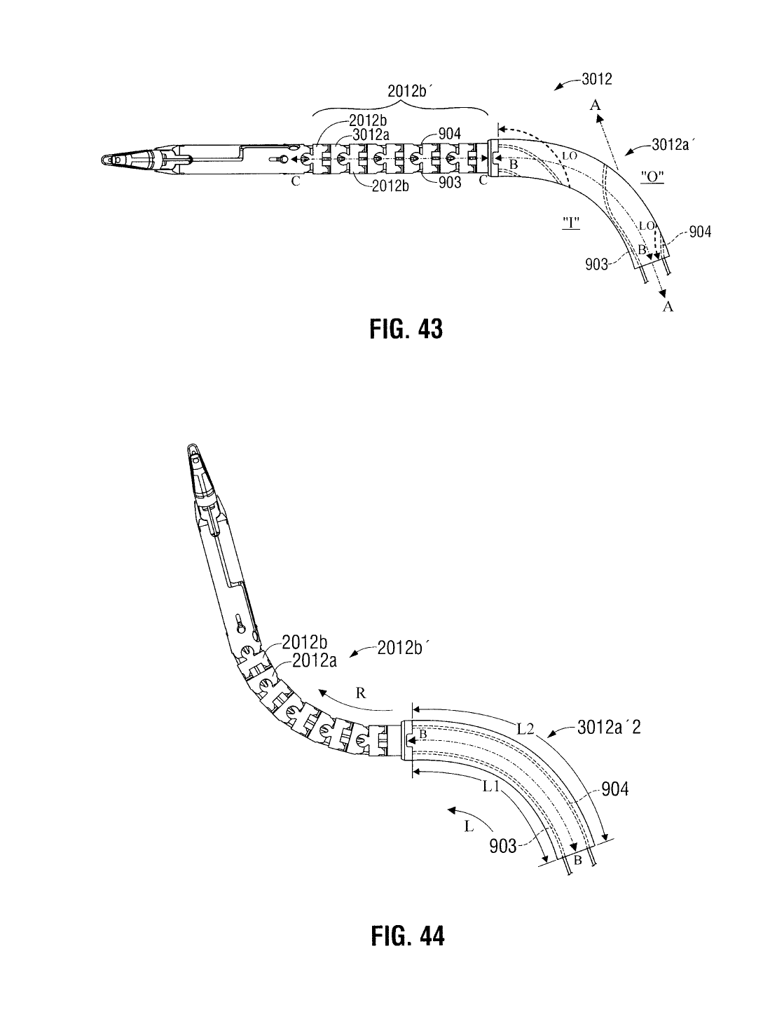

Referring now to FIG. 43, the flexible proximal portion 3012a′ of the elongated shaft 3012 may be shaped to assume a curve to the left from the perspective of a user. When such a curve is established, the proximal shaft axis B-B diverges from the longitudinal axis A-A. Due to the helical arrangement of the lumens 3311c and 3311d, however, the distal shaft axis C-C remains aligned with the proximal shaft axis B-B. This alignment is maintained since a length L0 of each of the cables 903 and 904 within the proximal portion 3012a′ remains constant as the proximal portion 3012a′ is curved. A portion of each of the cables 903 and 904 is disposed on a lateral side “O” of the axis B-B toward the outside of the curve, which is expanded as the proximal portion 3012a′ is curved. The length L0 of the cables does not increase, however. A portion of each of the cables 903 and 904 is also disposed on a lateral side “I” of the axis B-B toward the inside of the curve, which is compressed as the proximal portion 3012a′ is curved. The helical arrangement of the steering cables 903, 904 permits the expansion of the lateral side “O” to be offset by the compression of the lateral side “I” for each of the steering cables 903, 904.

In contrast to the proximal shaft portion 3012a′ having helical lumens 3311c, 3311d, proximal shaft portion 3012a′2 depicted in FIG. 44 provides a pair of non-helical lumens for the passage of steering cables 903 and 904. Steering cables 903 and 904 extend through the proximal shaft portion 3012a′2 in an axial direction, i.e., laterally offset from proximal shaft axis B-B. When the proximal shaft portion 3012a′2 is curved to the left, a first length L1 of steering cable 903 disposed within the proximal shaft portion 3012a′2 is reduced since steering cable 903 is disposed on a lateral side of the axis B-B toward the inside of the curve. A second length L2 of steering cable 904 is increased since steering cable 904 is disposed on a lateral side of the axis B-B toward the outside of the curve. To accommodate the increase of length L2, a portion of cable 904 is drawn into the proximal shaft portion 3012a′2 from the distal shaft portion 2012b′. To maintain the state of minimum stored energy in the steering cables 903, 904 associated with the “home” position wherein the collective stretching is least, the distal portion 2012b′ tends to curve to the right. In some instances, this response in the distal portion 2012b′ may frustrate the intent of a surgeon.

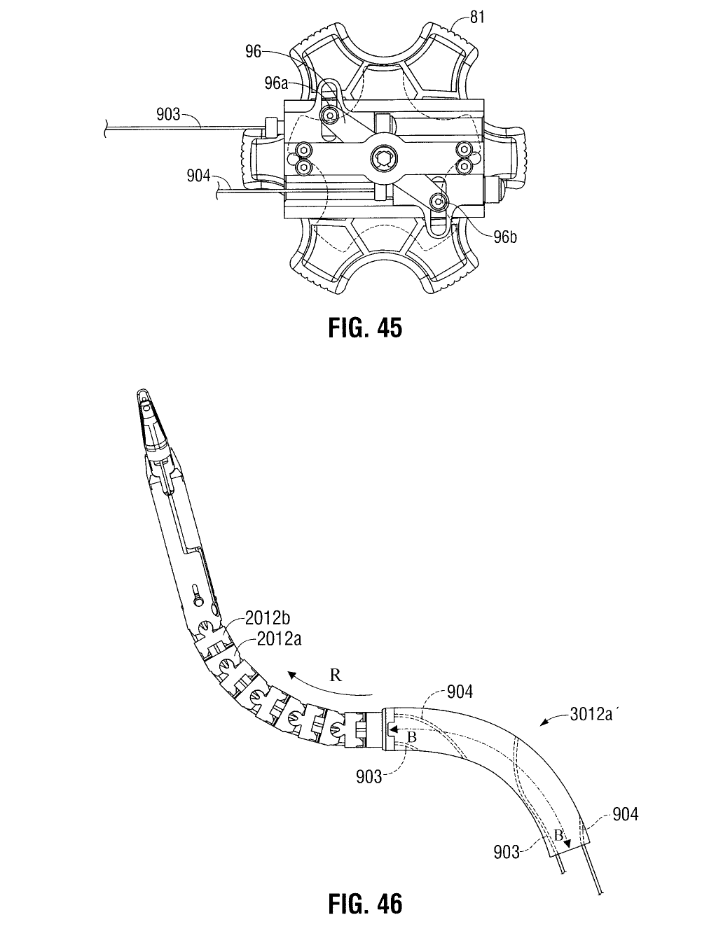

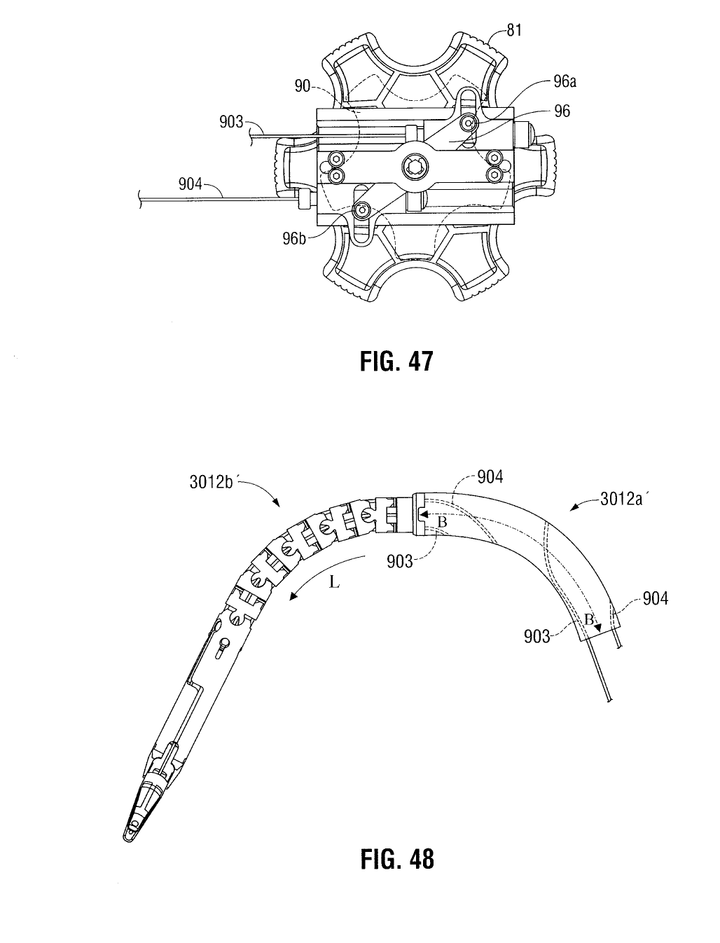

Referring now to FIGS. 45 and 46, the helical arrangement of steering cables 903, 904 permits articulation assembly 75 to be manipulated to articulate the end effector assembly 100 in the RIGHT and LEFT plane regardless of the curvature of the proximal portion 3012a′. As discussed above with reference to FIG. 5, the rotatable wheel 91 may be turned to move the steering cables 903 and 904. When the steering cable 904 is retracted proximally as depicted in FIG. 45, the end effector assembly 100 is articulated in the direction of arrow “R” with respect to the proximal shaft axis B-B as depicted in FIG. 46. The retraction of the steering cable 904 causes the links 2012a to pivot relative to neighboring links 2012b in the direction of arrow “R.” Similarly, rotatable wheel 91 may be turned to retract steering cable 903 as depicted in FIG. 47 and thus articulate the end effector 100 in the direction of arrow “L” as depicted in FIG. 48.

While several embodiments of the disclosure have been depicted in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An endoscopic surgical instrument for sealing tissue, comprising:
an end effector including a pair of jaw members adapted to connect to a source of electrosurgical energy, at least one jaw member of the pair of jaw members being movable relative to the other to move the end effector between an open configuration wherein the jaw members are substantially spaced for receiving tissue and a closed configuration wherein the jaw members are closer together for contacting the tissue;
a handle being manually movable to selectively induce motion in the end effector between the open configuration and the closed configuration; and
an elongated shaft defining a longitudinal axis and including distal and proximal ends, the distal end coupled to the end effector and the proximal end coupled to the handle, the elongated shaft including a plurality of links arranged sequentially such that neighboring links engage one another across a pair of rotational edges defined by each of the links to maintain the end effector in an aligned configuration with respect to the longitudinal axis, wherein each of the rotational edges is substantially spaced in a lateral direction from the longitudinal axis and wherein the neighboring links may pivot about the rotational edges to move the end effector to an articulated configuration.

2. The instrument according to claim 1, further comprising a pair of substantially elastic steering cables extending through at least one longitudinal cavity defined in the elongated shaft, the pair of steering cables coupled to a distal portion of the elongated shaft such that a differential tension in the pair of steering cables induces pivotal motion about the rotational edges to articulate the end effector in a first plane of articulation.

3. The instrument according to claim 2, wherein a general tension is imparted to the pair of steering cables when the end effector is in the aligned configuration.

4. The instrument according to claim 2, wherein the pair of rotational edges defined by one of the links is radially offset from the pair of rotational edges defined by another of the plurality of links by about 90° to define a second plane of articulation that is substantially orthogonal to the first plane of articulation.

5. The instrument according to claim 4, further comprising a second pair of steering cables extending through the least one longitudinal cavity and coupled to a distal portion of the elongated shaft such that a differential tension in the second pair of steering cables induces pivotal motion about the rotational edges to articulate the end effector in the second plane of articulation.

6. The instrument according to claim 1, wherein a substantially flat mating surface extends between the pair of rotational edges.

7. The instrument according to claim 1, wherein the rotational edges are rounded.

8. The instrument according to claim 1, wherein at least one of the plurality of links includes a rib extending therefrom to engage a neighboring link and thereby discourage radial displacement between the neighboring links.

9. An endoscopic surgical instrument for sealing tissue, comprising:
an end effector including a pair of jaw members adapted to connect to a source of electrosurgical energy, at least one jaw member of the pair of jaw members being movable relative to the other to move the end effector between an open configuration wherein the jaw members are substantially spaced for receiving tissue and a closed configuration wherein the jaw members are closer together for contacting the tissue;
a handle being manually movable to selectively induce motion in the end effector between the open configuration and the closed configuration; and
an elongated shaft defining a longitudinal axis and including distal and proximal ends, the distal end coupled to the end effector and the proximal end coupled to the handle, the elongated shaft including a flexible portion to permit the end effector to articulate, the flexible portion comprising:
a plurality of links arranged sequentially such that neighboring links engage one another across substantially flat forward and trailing mating faces to maintain the end effector in an aligned configuration with respect to the longitudinal axis, wherein at least one of the forward and trailing mating faces defines a rotational edge thereof about which the neighboring links may pivot to move the end effector to an articulated configuration;
at least one longitudinal cavity extending through the flexible portion of the elongated shaft; and
at least one steering cable extending through the at least one longitudinal cavity, the steering cable arranged to impart a compressive force on the plurality of links to maintain engagement between the mating faces.

10. The instrument according to claim 9, wherein the at least one of the forward and trailing mating faces defines a first pair of rotational edges on opposing sides of the longitudinal axis such that the end effector articulates in opposite directions in a first plane of articulation upon pivoting of the neighboring links about each of the first pair rotational edges.

11. The instrument according to claim 10, wherein at least one link of the plurality of links defines a second pair of rotational edges, the second pair of rotational edges oriented such that the end effector articulates in a second plane of articulation upon pivoting of neighboring links about the second first pair rotational edges, the second plane of articulation being substantially orthogonal to the first plane of articulation.

12. The instrument according to claim 11, wherein the at least one steering cable includes a first pair of steering cables coupled to a distal end of the elongated shaft such that relative longitudinal movement between the first pair of steering cables induces articulation of the end effector in the first plane of articulation.

13. The instrument according to claim 12, wherein the at least one steering cable further includes a second pair of steering cables coupled to a distal end of the elongated shaft such that relative longitudinal motion between the second pair of steering cables induces articulation of the end effector in the second plane of articulation.

14. The instrument according to claim 11, wherein each link of the plurality of links is similar in construction and each link is oriented with a 90° offset with respect to neighboring links to orient the pair of rotational edges.

15. The instrument according to claim 9, wherein at least one of the plurality of links includes a rib extending therefrom to engage a neighboring link and thereby discourage radial displacement between the neighboring links.

16. The instrument according to claim 9, wherein at least one steering cable is substantially elastic.

17. An endoscopic surgical instrument for sealing tissue, comprising:
an end effector including a pair of jaw members adapted to connect to a source of electrosurgical energy, at least one jaw member of the pair of jaw members being movable relative to the other to move the end effector between an open configuration wherein the jaw members are substantially spaced for receiving tissue and a closed configuration wherein the jaw members are closer together for contacting the tissue;
a handle being manually movable to selectively induce motion in the end effector between the open configuration and the closed configuration; and
an elongated shaft defining a longitudinal axis and including distal and proximal ends, the distal end coupled to the end effector and the proximal end coupled to the handle, the elongated shaft including a plurality of links arranged sequentially such that each of the links may pivot relative to a neighboring link to move the end effector between an aligned configuration and an articulated configuration with respect to the longitudinal axis, wherein each of the links includes a substantially rigid base and a pair of relatively flexible tubes extending therefrom to engage the neighboring link.

18. The instrument according to claim 17, further comprising a pair of substantially elastic steering cables extending through at least one longitudinal cavity defined in the elongated shaft, the pair of steering cables coupled to a distal portion of the elongated shaft such that a differential tension in the pair of steering cables induces elastic bending in the pair of flexible tubes to articulate the end effector in a first plane of articulation.

19. The instrument according to claim 18, wherein a general tension is imparted to the pair of steering cables when the end effector is in the aligned configuration.

20. The instrument according to claim 18, wherein the pair of flexible tubes defined by one of the links is radially offset from the pair of flexible tubes defined by another of the plurality of links by about 90° to define a second plane of articulation that is substantially orthogonal to the first plane of articulation.

21. The instrument according to claim 20, further comprising a second pair of steering cables extending through the least one longitudinal cavity and coupled to a distal portion of the elongated shaft such that a differential tension in the second pair of steering cables induces bending of the flexible tubes to articulate the end effector in the second plane of articulation.

22. The instrument according to claim 20, wherein the at least one longitudinal cavity extends through the flexible tubes.

23. The instrument according to claim 17, wherein the flexible tubes include a nitinol alloy.

24. The instrument according to claim 17, wherein at least one of the plurality of links includes a rib extending therefrom to engage a neighboring link and thereby discourage radial displacement between the neighboring links.

25. An endoscopic surgical instrument for sealing tissue, comprising:
an end effector including a pair of jaw members adapted to connect to a source of electrosurgical energy, at least one jaw member of the pair of jaw members being movable relative to the other to move the end effector between an open configuration wherein the jaw members are substantially spaced for receiving tissue and a closed configuration wherein the jaw members are closer together for contacting the tissue;
a handle being manually movable to selectively induce motion in the end effector between the open configuration and the closed configuration; and
an elongated shaft defining a longitudinal axis and including distal and proximal ends, the distal end coupled to the end effector and the proximal end coupled to the handle, the elongated shaft including a flexible portion to permit the end effector to articulate, the flexible portion comprising:
a plurality of links wherein at least one link includes a substantially rigid base and at least one relatively flexible tube extending therefrom to engage a neighboring link and maintain the end effector in an aligned configuration with respect to the longitudinal axis;
at least one longitudinal cavity extending through the flexible tube; and
at least one steering cable extending through the at least one longitudinal cavity, the steering cable arranged to impart a compressive force on the plurality of links to induce bending in the flexible tube to move the end effector to an articulated configuration.

26. The instrument according to claim 25, wherein the at least one flexible tube includes a first pair of flexible tubes disposed on opposing sides of the longitudinal axis to define a first plane of articulation such that the end effector articulates in opposite directions in the first plane of articulation upon bending of the flexible tubes.

27. The instrument according to claim 16, wherein the neighboring link includes second pair of flexible tubes disposed on opposing sides of the longitudinal axis to define a second plane of articulation such that the end effector articulates in the second plane of articulation upon bending of the flexible tubes, the second plane of articulation being substantially orthogonal to the first plane of articulation.

28. The instrument according to claim 27, wherein the at least one steering cable includes a first pair of steering cables coupled to a distal end of the elongated shaft such that relative longitudinal movement between the first pair of steering cables induces articulation of the end effector in the first plane of articulation.

29. The instrument according to claim 28, wherein the at least one steering cable further includes a second pair of steering cables coupled to a distal end of the elongated shaft such that relative longitudinal motion between the second pair of steering cables induces articulation of the end effector in the second plane of articulation.

30. The instrument according to claim 27, wherein each link of the plurality of links is similar in construction and each link is oriented with a 90° offset with respect to neighboring links to orient the pair flexible tubes.

31. The instrument according to claim 25, wherein at least one of the plurality of links includes a rib extending therefrom to engage a neighboring link and thereby discourage radial displacement between the neighboring links.

32. The instrument according to claim 31, wherein the at least one link includes a proximal rib projecting from the rigid base thereof to engage a distal rib projecting from a rigid base of the neighboring link.

33. The instrument according to claim 32, wherein the proximal rib engages the distal rib across a substantially flat sliding face.

34. The instrument according to claim 25, wherein at least one steering cable is substantially elastic.

35. The instrument according to claim 25, wherein the at least one flexible tube includes a nitinol alloy.

36. An endoscopic surgical instrument comprising:
an end effector including a pair of jaw members, at least one jaw member of the pair of jaw members being movable relative to the other to move the end effector between an open configuration wherein the jaw members are substantially spaced for receiving tissue and a closed configuration wherein the jaw members are closer together for contacting the tissue;
a handle being manually movable to selectively induce motion in the end effector between the open configuration and the closed configuration;
an elongated shaft defining a longitudinal axis and including distal and proximal ends, the distal end coupled to the end effector and the proximal end coupled to the handle, the elongated shaft including a flexible portion movable to a non-aligned configuration with respect to the longitudinal axis, the flexible portion exhibiting a composite construction comprising:
an outer tubular layer defining a first wall thickness; and
an inner tubular layer extending axially through the outer tubular layer, the inner tubular layer defining a second wall thickness;
wherein the inner tubular layer is relatively rigid with respect to the outer tubular layer, and wherein the first wall thickness is relatively thick with respect to the second wall thickness.

37. The instrument according to claim 36, wherein the outer tubular layer exhibits a modulus of elasticity of about 52,600 psi.

38. The instrument according to claim 37, wherein the outer tubular layer comprises a thermoplastic elastomer.

39. The instrument according to claim 36, wherein the inner tubular layer exhibits a modulus of elasticity of about 6×10⁶ psi.

40. The instrument according to claim 39, wherein the inner tubular layer comprises a nitinol tube.

41. The instrument according to claim 39, wherein the inner tubular layer comprises a stainless steel tube.

42. The instrument according to claim 41, wherein the stainless steel tube comprises lateral notches formed therein to facilitate lateral bending of the flexible portion of the elongated shaft.

43. The instrument according to claim 42, wherein the lateral notches are arranged in a helical pattern along a length of the stainless steel tube.

44. The instrument according to claim 36, wherein the flexible portion exhibits an axial rigidity of about 20,000 lb and flexural rigidity of about 60 lb·².

45. The instrument according to claim 36, wherein the second wall thickness is about 5-15 percent of the first wall thickness.

46. The instrument according to claim 36, wherein the flexible portion exhibits sufficient axial rigidity to maintain a shape and orientation of the flexible portion in a non-aligned configuration with respect to the longitudinal axis during normal surgical use of the instrument.

47. The instrument according to claim 46, wherein the flexible portion includes at least one passageway defined therein, and wherein the instrument includes at least one tensile member extending through the passageway and coupled to the end effector, the at least one tensile member being movable to induce motion in the end effector.

48. The instrument according to claim 47, wherein the elongated shaft includes an articulating portion movable between an aligned configuration and an articulated configuration with respect to the flexible portion.

49. The instrument according to claim 48, wherein the at least one tensile member includes a pair of steering cables coupled to the end effector such that a differential tension in the pair of steering cables induces articulation of the end effector in a first plane of articulation.

50. The instrument according to claim 49, wherein a general tension is imparted to the pair of steering cables when the end effector is in the aligned configuration.

51. The instrument according to claim 48, wherein the articulating portion includes a plurality of links arranged sequentially such that each of the links may pivot relative to a neighboring link to move the articulating portion between the aligned and articulated configurations.

52. The instrument according to claim 51, wherein a first pivoting axis defined by one of the links is radially offset from a second pivoting axis defined by another of the plurality of links by about 90° to define a second plane of articulation that is substantially orthogonal to the first plane of articulation.

53. The instrument according to claim 49, further comprising a second pair of steering cables extending through the least one passageway and coupled to the end effector such that a differential tension in the second pair of steering cables pivots the links about the second pivoting axis to induce articulation of the end effector in the second plane of articulation.

54. An endoscopic surgical instrument comprising:
an end effector including a pair of jaw members, at least one jaw member of the pair of jaw members being movable relative to the other to move the end effector between an open configuration wherein the jaw members are substantially spaced for receiving tissue and a closed configuration wherein the jaw members are closer together for contacting the tissue;
a handle being manually movable to selectively induce motion in the end effector between the open configuration and the closed configuration; and
an elongated shaft defining a longitudinal axis and including distal and proximal ends, the distal end coupled to the end effector and the proximal end coupled to the handle, the elongated shaft including a flexible portion to permit the end effector to articulate with respect to the longitudinal axis, the flexible portion including an anisotropic tube, the tube exhibiting a modulus of elasticity that generally decreases as a function of radius.

55. An endoscopic surgical instrument for sealing tissue, comprising:
an end effector including a pair of jaw members adapted to connect to a source of electrosurgical energy, at least one jaw member of the pair of jaw members being movable relative to the other to move the end effector between an open configuration wherein the jaw members are substantially spaced for receiving tissue and a closed configuration wherein the jaw members are closer together for contacting the tissue;
a handle being manually movable to selectively induce motion in the end effector between the open configuration and the closed configuration; and
an elongated shaft defining a longitudinal axis and including distal and proximal ends, the distal end coupled to the end effector and the proximal end coupled to the handle, the elongated shaft including a flexible portion movable to a non-aligned configuration with respect to the longitudinal axis, the flexible portion defining at least one helical passageway therethrough; and
at least one tensile member extending through the helical passageway and coupled to the end effector, the at least one tensile member movable to induce motion in the end effector.

56. The instrument according to claim 55, wherein the at least one helical passageway traverses a radial arc of about 360 degrees.

57. The instrument according to claim 55, wherein the at least one helical passageway defines a helical lumen extending through an interior of the flexible portion of the elongated shaft.

58. The instrument according to claim 55, wherein the flexible portion exhibits sufficient rigidity to maintain a shape and orientation of the flexible portion in a non-aligned configuration with respect to the longitudinal axis during normal surgical use of the instrument.

59. The instrument according to claim 58, wherein the flexible portion includes a composite of a flexible tube and a rigidizing element.

60. The instrument according to claim 55, wherein the elongated shaft includes an articulating portion movable between an aligned configuration and an articulated configuration with respect to the flexible portion.

61. The instrument according to claim 60, wherein the at least one tensile member includes a pair of steering cables coupled to the end effector such that a differential tension in the pair of steering cables induces articulation of the end effector in a first plane of articulation.

62. The instrument according to claim 61, wherein a general tension is imparted to the pair of steering cables when the end effector is in the aligned configuration.

63. The instrument according to claim 61, wherein the articulating portion includes a plurality of links arranged sequentially such that each of the links may pivot relative to a neighboring link to move the articulating portion between the aligned and articulated configurations.

64. The instrument according to claim 63, wherein a first pivoting axis defined by one of the links is radially offset from a second pivoting axis defined by another of the plurality of links by about 90° to define a second plane of articulation that is substantially orthogonal to the first plane of articulation.

65. The instrument according to claim 64, further comprising a second pair of steering cables extending through the least one helical passageway and coupled to the end effector such that a differential tension in the second pair of steering cables pivots the links about the second pivoting axis to induce articulation of the end effector in the second plane of articulation.

66. An endoscopic surgical instrument for sealing tissue, comprising:
an end effector including a pair of jaw members adapted to connect to a source of electrosurgical energy, at least one jaw member of the pair of jaw members being movable relative to the other to move the end effector between an open configuration wherein the jaw members are substantially spaced for receiving tissue and a closed configuration wherein the jaw members are closer together for contacting the tissue;
a handle being manually movable to selectively induce motion in the end effector between the open configuration and the closed configuration;
an elongated shaft defining a longitudinal axis and including distal and proximal ends, the distal end coupled to the end effector and the proximal end coupled to the handle, the elongated shaft including a flexible portion to permit the end effector to articulate with respect to the longitudinal axis, the flexible portion defining a shaft axis extending centrally therethrough, the flexible portion including a passageway extending therethrough, the passageway including a first longitudinal length disposed on a first lateral side of the shaft axis and a second longitudinal length disposed on an opposed lateral side of the shaft axis; and
a tensile member extending through the passageway and coupled to the end effector such that longitudinal motion of the tensile member induces motion in the end effector.

67. The instrument according to claim 66, wherein the passageway is helically arranged through the flexible portion.

68. The instrument according to claim 67, wherein the first and second longitudinal lengths are about equal with respect to one another.

69. The instrument according to claim 67, wherein the passageway defines a groove on an exterior surface of a tubular member.

70. The instrument according to claim 66, wherein the elongated shaft includes an articulating portion movable between an aligned configuration and an articulated configuration with respect to the longitudinal axis, and wherein the longitudinal motion of the tensile member induces movement of the articulation portion between the aligned and articulated configurations.

71. The instrument according to claim 70, wherein the tensile member is substantially elastic.

72. The instrument according to claim 70, wherein the articulating portion includes a plurality of links arranged sequentially such that each of the links may pivot relative to a neighboring link to move the articulating portion between the aligned and articulated configurations.
